(19) United States
(12) Patent Application Publication
(10) Pub. No.: US 2006/0004037 A1
(43) Pub. Date: Jan. 5, 2006

(54) Novel tricyclic compounds and related methods of treatment

(75) Inventors: Sherry Morissette, Arlington, MA (US); Michael Read, Burke, VA (US)

Correspondence Address: TRANSFORM PHARMACEUTICALS, INC., 29 HARTWELL AVENUE, LEXINGTON, MA, 02421, US

(73) Assignee: TransForm Pharmaceuticals, Inc., Lexington, MA (US)

(21) Appl. No.: 11/088,469

(22) Filed: Mar. 24, 2005

Related U.S. Application Data
(60) Provisional application No. 60/556,407, filed on Mar. 25, 2004.
(60) Provisional application No. 60/610,295, filed on Sep. 16, 2004.

(51) Int. Cl. A61K 31/4745; C07D 491/02

(52) U.S. Cl. 514/291; 546/83

(57) ABSTRACT

The invention provides novel lipid soluble forms of tricyclic antineoplastic compounds. These forms include salts, co-crystals, and solvates of the tricyclic antineoplastic compounds. The invention also provides novel pharmaceutical compositions comprising these novel lipid soluble forms and related methods of treatment. Compositions and methods of the invention are useful in the treatment of neoplasms, including MRP-1-related resistant neoplasms.

### CROSS-REFERENCE TO RELATED APPLICATION

The present application claims the benefit of priority of U.S. Provisional Application Ser. No. 60/556,407, filed Mar. 25, 2004 and U.S. Provisional Application Ser. No. 60/610,295, filed Sep. 16, 2004, which are hereby incorporated by reference herein in their entirety, including any figures, tables or drawings.

### FIELD OF THE INVENTION

The invention provides novel lipid soluble forms of tricyclic antineoplastic compounds. These forms include salts, co-crystals, and solvates of the tricyclic antineoplastic compounds.

The invention also provides novel pharmaceutical compositions comprising these novel lipid soluble forms and related methods of treatment.

Compositions and methods of the invention are useful in the treatment of neoplasms, including MRP-1-related resistant neoplasms.

### BACKGROUND OF THE INVENTION

Several types of cancer are now considered to be curable by chemotherapy. These cancers include Hodgkin's disease, large cell lymphoma, acute lymphocytic leukemia, testicular cancer and early stage breast cancer. Other cancers such as ovarian cancer, small cell lung and advanced breast cancer, while not yet curable, have exhibited a positive response to combination chemotherapy.

Drug resistance remains problematic in cancer treatment. Drug resistance includes both intrinsic resistance at the time of treatment using chemotherapy and acquired drug resistance. Combination chemotherapies employed in the treatment of drug resistant cancers seek to avoid propagation of additional resistant cells and to kill existing resistant cells.

After selection for resistance to a single cytotoxic drug, cells may become cross resistant to a whole range of drugs with different structures and cellular targets, e.g., alkylating agents, antimetabolites, hormones, platinum-containing drugs, and natural products. This phenomenon is known as multidrug resistance (MDR). In some types of cells, this resistance is inherent, while in others, such as small cell lung cancer, it is usually acquired. Such resistance is known to be multifactorial and is conferred by at least two proteins: the 170 kDa P-glycoprotein (MDR1) and the more recently identified 190 kDa multidrug resistance protein (MRP1). Although both MDR1 and MRP1 belong to the ATP-binding cassette superfamily of transport proteins, they are structurally very different molecules and share less than 15% amino acid homology.

U.S. Pat. No. 6,673,809 (“809 Patent”) describes tricyclic compounds that are selective inhibitors of multidrug resistant protein (MRP1) and are thus useful in treating MRP1 conferred MDR in a resistant neoplasm and a neoplasm susceptible to resistance. For example, compounds of the '809 patent are effective in the treatment of a resistant neoplasm or a neoplasm susceptible to resistance that is associated with non-small cell lung, breast, gastric, refractory prostate, leukemia, and colorectal cancers.

Notwithstanding this efficacy, it has proven difficult to formulate compounds described in the '809 patent in clinically useful dosage forms. In particular, it has proven difficult to formulate compounds described in the '809 patent in oral dosage forms that achieve pharmacokinetic profiles associated with the optimal treatment of a resistant neoplasm or a neoplasm susceptible to resistance. Formulation vehicles tested with '809 patent Compounds have failed to achieve requisite area under the curve (“AUC”) values at acceptable excipient levels.

Accordingly, the need exists for pharmaceutical compositions that comprise compounds described in the '809 patent and that are effective in the treatment of a resistant neoplasm or a neoplasm susceptible to resistance. These pharamaceutical compositions should achieve requisite area under the curve (“AUC”) values at acceptable excipient levels, and ideally should also exhibit the following properties: poor aqueous solubility or dissolution characteristics; a rate of in vivo absorption that is not limited by permeability; bioavailability that is not limited by metabolism; and, perhaps, a crystalline or amorphous solid form.

### SUMMARY OF THE INVENTION

The invention provides novel lipid soluble forms, including salts, co-crystals, and solvates, of antineoplastic compounds of the formula (I): 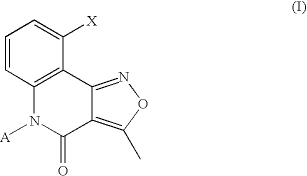
wherein:
A is substituted or unsubstituted and is 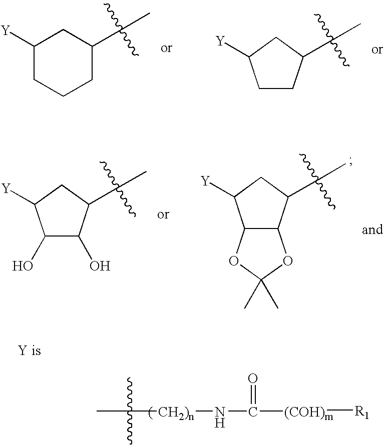
   where n and m can be the same or different and are individually 0 or 1, R₁ is substituted or unsubstituted benzene or pyridine, X is a halogen, and the compound of formula (I) is in racemic or enantiomeric form or is an anomer.

The invention also provides novel pharmaceutical compositions comprising novel lipid soluble forms and related methods of treatment. Pharmaceutical compositions of the invention can be used to treat MRP-1-related resistant neoplasms.

Embodiments of the invention can be made by crystallizing or recrystallizing a compound of formula (I) in a crystallization solvent comprising a variety of inorganic and organic acids. These organic acids may be racemic or substantially enantiomerically pure.

The invention also provides novel lipid soluble forms, including salts, co-crystals, and solvates, of antineoplastic compounds of the formula (II) (the compound of formula (II), both in racemic and enantiomeric form, is designated herein as LSN 487355):

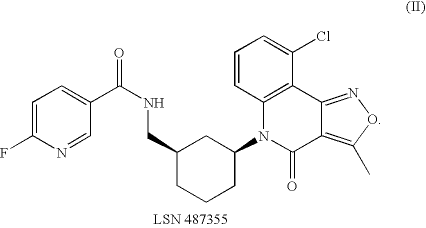

The invention also provides novel lipid soluble forms, including salts, co-crystals, and solvates, of antineoplastic compounds of the formula (III) (the compound of formula (III), both in racemic and enantiomeric form, is designated herein as LSN 509207):

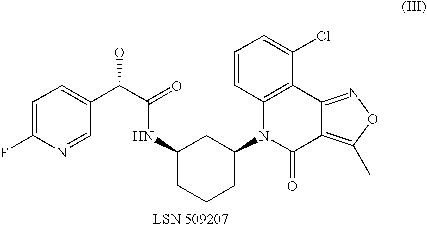

In another embodiment, the invention provides novel liquid formulations comprising an antineoplastic effective amount of a form of LSN 487355 or LSN 509207 and an excipient system comprising Labrasol, wherein the excipient system comprises approximately 25% to 75% by weight of the formulation, the lipid solubility of the compound in the excipient system is approximately 10 mg/ml to approximately 150 mg/ml, and the formulation can be administered orally or parenterally.

The invention provides a variety of novel pharmaceutical compositions comprising novel lipid soluble forms of LSN 487355 or LSN 509207 and related methods of treatment. Pharmaceutical compositions of the invention can be used to treat neoplasms, including MRP-1-related resistant neoplasms.

These and other embodiments of the invention are described further in the detailed description of the invention.

### BRIEF DESCRIPTION OF THE FIGURES

FIG. 1 shows a TGA thermogram for the LSN 487355 phosphate salt.

FIG. 2 shows a DSC thermogram for the LSN 487355 phosphate salt.

FIG. 3 shows a PXRD diffractogram for the LSN 487355 phosphate salt.

FIG. 4 shows a PXRD diffractogram for the nitromethane solvate of LSN 487355 phosphate salt.

FIG. 5 shows a PXRD diffractogram for the nitromethane solvate of LSN 487355 oxalate salt.

FIG. 6 shows a PXRD diffractogram for the dinitromethane solvate of LSN 487355 oxalate salt.

FIG. 7 shows a PXRD diffractogram for the LSN 487355 nitrate salt.

FIG. 8 shows a PXRD diffractogram for the LSN 487355 form from methanol in the presence of strong acid.

FIG. 9 shows a PXRD diffractogram for the LSN 487355:fumaric acid co-crystal (bottom).

FIG. 10 shows a PXRD diffractogram for the LSN 487355 DMSO solvate.

FIG. 11 shows a PXRD diffractogram for the LSN 487355 methyl tert-butyl ether solvate.

FIG. 12 shows a PXRD diffractogram for the LSN 487355 formamide solvate.

FIG. 13 shows a solubility comparison of LSN 487355 free base and the phosphate salt in several excipients.

FIG. 14 shows the kinetic dissolution of several liquid formulations of LSN 487355 in simulated gastric fluid (SGF).

FIG. 15 shows a TGA thermogram for the LSN 509207 Form II polymorph.

FIG. 16 shows a DSC thermogram (solid/top line) for the LSN 509207 Form II polymorph.

FIG. 17 shows a PXRD diffractogram for the LSN 509207 Form II polymorph.

FIG. 18 shows a TGA thermogram (dashed line) for the LSN 509207 Form III polymorph.

FIG. 19 shows a DSC thermogram (solid/bottom line) for the LSN 509207 Form III polymorph.

FIG. 20 shows a PXRD diffractogram for the LSN 509207 Form III polymorph.

FIG. 21 shows a PXRD diffractogram for the LSN 509207 Form III polymorph.

FIG. 22 shows a TGA thermogram (solid/top line) for the LSN 509207 Form IV polymorph.

FIG. 23 shows a DSC thermogram (solid/bottom line) for the LSN 509207 Form IV polymorph.

FIG. 24 shows a PXRD diffractogram (bottom) for the LSN 509207 Form IV polymorph.

FIG. 25 shows a PXRD diffractogram for the LSN 509207 Form IV polymorph.

FIG. 26 shows a TGA thermogram (solid line) for the LSN 509207 Form V polymorph.

FIG. 27 shows a DSC thermogram (solid/bottom line) for the LSN 509207 Form V polymorph.

FIG. 28 shows a PXRD diffractogram for the LSN 509207 Form V polymorph.

FIG. 29 shows a TGA thermogram for the LSN 509207:1-hydroxy-2-naphthoic acid co-crystal.

FIG. 30 shows a DSC thermogram (dashed/bottom line) for the LSN 509207:1-hydroxy-2-naphthoic acid co-crystal.

FIG. 31 shows a PXRD diffractogram for the LSN 509207:1-hydroxy-2-naphthoic acid co-crystal.

FIG. 32 shows a PXRD diffractogram for the LSN 509207 tetrahydrofuran solvate.

FIG. 33 shows a PXRD diffractogram for the LSN 509207 acetic acid solvate.

FIG. 34 shows a PXRD diffractogram for the LSN 509207 pyridine solvate.

FIG. 35 shows a PXRD diffractogram for the LSN 509207 dioxane solvate.

FIG. 36 shows a PXRD diffractogram for the LSN 509207 nitromethane solvate.

FIG. 37 shows a PXRD diffractogram for the LSN 509207 methylene chloride solvate.

### DETAILED DESCRIPTION OF THE INVENTION

As used herein, the following terms have the following respective meanings.

A “solvate” is a complex of variable stoichiometry formed by a solute (either a compound of formula (I), including LSN 487355 and LSN 509207 or a salt, co-crystal, hydrate, or polymorph of LSN 487355 or LSN 509207) and a liquid at room temperature (about 22 degrees C.), including but not limited to an alcohol (for example, methanol or ethanol), naphthalene, dimethyl sulfoxide, methyl tert-butyl ether, formamide, acetonitrile, nitromethane, methylene chloride, acetic acid, pyridine, 1,4-dioxane, tetrahydrofuran, and 1,2-dichloroethane.

“Organic or inorganic acids” include, but are not limited to, carboxylic acids, dicarboxylic acids, hydrochloric acid, phosphoric acid, sulfuric acid, benzenesulfonic acid, methanesulfonic acid, and, in general terms, any acidic species that will form a thermodynamically stable crystalline (salt) form upon reaction with a free base comprising a compound of formula (I). For example, organic or inorganic acids include those that form a thermodynamically stable crystalline (salt) form upon reaction with LSN 487355 and LSN 509207.

“Carboxylic acids” include, but are not limited to, formic, acetic, propionic, butyric, isobutyric, valeric, isovaleric, pivalic, caproic, caprylic, capric, lauric, myristic, palmitic, stearic, acrylic, crotonic, benzoic, cinnamic, and salicylic acids.

“Dicarboxylic acid” means a compound of formula (IV): 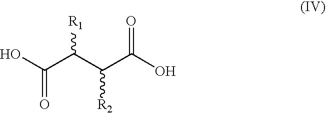
wherein R₁ and R₂ are each independently H, OH, Cl, Br, I, substituted or unsubstituted C₁₋₆ alkyl, substituted or unsubstituted aryl or R₁ and R₂ taken together represent a double bond as well as stereochemically pure D or L salts of a compound of formula (IV).

Examples of the dicarboxylic acid of formula (IV) include but are not limited to oxalic acid, succinic acid, maleic acid, tartaric acid, malic acid or fumaric acid. Dicarboxylic acids of formula (IV) that can be used to make compounds of the invention include succinic acid, tartaric acid, malic acid, and fumaric acid. Dicarboxylic acids such as malonic acid and adipic acid can also be used. Dicarboxylic acids can be in the form of a substantially pure (R)(+) enantiomer; a substantially pure (R)(−) enantiomer; a substantially pure (S)(+) enantiomer; or a substantially pure (S)(−) enantiomer.

“Co-crystal” as used herein means a crystalline material comprised of two or more unique solids at room temperature, each containing distinctive physical characteristics, such as structure, melting point, and heats of fusion, with the exception that, if specifically stated, the API (active pharmaceutical ingredient) may be a liquid at room temperature. The co-crystals of the present invention comprise a co-crystal former H-bonded to LSN 487355 or LSN 509207 or a derivative thereof. The co-crystal former may be H-bonded directly to LSN 487355 or LSN 509207 or may be H-bonded to an additional molecule which is bound to LSN 487355 or LSN 509207. The additional molecule may be H-bonded to LSN 487355 or LSN 509207 or bound ionically or covalently to LSN 487355 or LSN 509207. The additional molecule could also be a different API. Solvates of LSN 487355 or LSN 509207 compounds that do not further comprise a co-crystal former are not co-crystals according to the present invention. The co-crystals may however, include one or more solvate molecules in the crystalline lattice. That is, solvates of co-crystals, or a co-crystal further comprising a solvent or compound that is a liquid at room temperature, is included in the present invention, but crystalline material comprised of only LSN 487355 or LSN 509207 and one or more liquids (at room temperature) are not included. Other modes of molecular recognition may also be present including, pi-stacking, guest-host complexation and van der Waals interactions. Of the interactions listed above, hydrogen-bonding is the dominant interaction in the formation of the co-crystal, (and a required interaction according to the present invention) whereby a non-covalent bond is formed between a hydrogen bond donor of one of the moieties and a hydrogen bond acceptor of the other.

Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads. An alternative embodiment provides for a co-crystal wherein the co-crystal former is a second API. In another embodiment, the co-crystal former is not an API. For purposes of the present invention, the chemical and physical properties of LSN 487355 or LSN 509207 in the form of a co-crystal may be compared to a reference compound that is LSN 487355 or LSN 509207 in a different form. The reference compound may be specified as a free form, or more specifically, an anhydrate or hydrate of a free form, or more specifically, for example, a hemihydrate, monohydrate, dihydrate, trihydrate, quadrahydrate, pentahydrate; or a solvate of a free form. For example, the reference compound for LSN 487355 or LSN 509207 in free form co-crystallized with a co-crystal former can be LSN 487355 or LSN 509207 in free form. The reference compound may also be specified as crystalline or amorphous. The reference compound may also be specified as the most stable polymorph of the specified form of the reference compound.

“Halo” and “halogen” are used in the conventional sense to refer to a chloro, bromo, fluoro or iodo substituent.

“Lipid soluble forms” encompass, but are not limited to, co-crystals, salts, hydrates, and solvates that have lipid solubility values greater than 10 micrograms/mL, greater than 25 micrograms/mL, greater than 50 micrograms/mL, greater than 75 micrograms/mL, greater than 100 micrograms/mL, greater than 150 micrograms/mL, or greater than 250 micrograms/mL in a solution with a pH of about 1. Lipid soluble forms can comprise forms obtained by the crystallization or recrystallization of a salt comprising the reaction product of a compound of formula (I) and an organic acid or an inorganic acid in a crystallization solvent that is present in either a stoichiometric or non-stoichiometric ratio relative to the salt. Lipid soluble forms can also comprise LSN 487355 and LSN 509207 salts that are crystallized or recrystallized in a crystallization solvent as defined herein.

The term “anomer” as used herein means one of a pair of isomers of a cyclic compound resulting from creation of a new point of symmetry when a rearrangement of atoms occurs at an aldehyde or ketone position.

As used herein, the terms “stereoisomer” or “stereoisomeric form” means compounds having a stereoisomeric purity of at least 90%, 95%, or up to a stereoisomeric purity of 100% by weight, for example, compounds having a stereoisomeric purity of at least 97% up to a stereoisomeric purity of 100%, or having a stereoisomeric purity of at least 99% up to a stereoisomeric purity of 100% by weight, said weight percent based upon the total weight of the desired stereoisomers of the compound.

“Alkyl” means a straight chain or branched, saturated or unsaturated alkyl, cyclic or non-cyclic hydrocarbon having from 1 to 10 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (also referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cycloalkyls are also referred to herein as “carbocyclic” rings systems, and include bi- and tri-cyclic ring systems having from 8 to 14 carbon atoms such as a cycloalkyl (such as cyclopentane or cyclohexane) fused to one or more aromatic (such as phenyl) or non-aromatic (such as cyclohexane) carbocyclic rings.

As used herein, the term “aryl” means a carbocyclic or heterocyclic aromatic group containing from 5 to 10 ring atoms. The ring atoms of a carbocyclic aromatic group are all carbon atoms, and include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. A carbocyclic aromatic group can be unsubstituted or substituted. For example, the carbocyclic aromatic group can be a phenyl group. The ring atoms of a heterocyclic aromatic group contains at least one heteroatom, for example, 1 to 3 heteroatoms, independently selected from nitrogen, oxygen, and sulfur. Illustrative examples of heterocyclic aromatic groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, indolyl, oxetanyl, azepinyl, piperazinyl, morpholinyl, dioxanyl, thietanyl and oxazolyl. A heterocyclic aromatic group can be unsubstituted or substituted. For example, a heterocyclic aromatic can be a monocyclic ring, wherein the ring comprises 2 to 5 carbon atoms and 1 to 3 heteroatoms.

The term “substituted” as used herein means any of the above groups (i.e., aryl or alkyl) wherein at least one hydrogen atom is replaced with a substituent. In the case of a keto substituent (C═O) two hydrogen atoms are replaced. Substituents include halogen, hydroxy, alkyl, aryl, arylalkyl, heterocycle or heterocyclealkyl.

“LSN 487355” means a compound of formula (II), and racemates, enantiomers, and anomers thereof.

“LSN 509207” means a compound of formula (III), and racemates, enantiomers, and anomers thereof.

As used herein, the term “adjunctively administered” refers to the administration of one or more compounds or active ingredients in addition to pharmaceutically acceptable salt, solvate, or polymorph of a racemate or stereoisomer of a compound of the formula (I), such as LSN 487355 or LSN 509207, either simultaneously with the same or at intervals prior to, during, or following administration of the pharmaceutically acceptable salt, solvate, or polymorph of a racemate or stereoisomer of a compound of the formula (I), such as LSN 487355 or LSN 509207, to achieve the desired therapeutic or prophylactic effect.

A “polymorph” is a particular crystalline form of an organic compound that exists in a variety of crystal structures. While polymorphic modifications have the same chemical composition, they differ in packing, geometrical arrangement, and other descriptive properties of the crystalline solid state. As such, these modifications may have different solid-state physical properties such as shape, color density, hardness, deformability, stability, and dissolution properties.

As used herein, the term “pharmaceutically acceptable form” refers to a form, e.g., a salt, prepared from pharmacologically acceptable anions, which include but are not limited to anions such as hydrochloride, phosphate, formate, oxalate, adipate, succinate, fumarate, malate, tartrate, malonate, maleate, mesylate and benzenesulfonate. Pharmacologically acceptable anions also include, but are not limited to, oxalate, tartrate, benzenesulfonate, malate and succinate, hydrobromide, bitartrate, para-toluenesulfonate, glycolate, glucuronate, mucate, gentisate, isonicotinate, saccharate, acid phosphate, hydroiodide, nitrate, sulfate, bisulfate, acetate, propionate, camphorsulfonate, gluconate, isothionate, lactate, furoate, glutamate, ascorbate, benzoate, anthranilate, salicylate, phentylacetate, mandelate, embonate (pamoate), methanesulfonate, ethanesulfonate, pantothenate, stearate, sulfanilate, alginate, p-toluenesulfonate, mesylate, and galacturonate.

A phosphate salt of LSN 487355 is a specific embodiment and is described in detail hereinafter.

“Crystallization solvents” include aromatic hydrocarbons, C₃-C₉ ketones, C₃-C₉ branched alcohols, C₃-Cg esters, C₅-C₉ hydrocarbons, C₃-Cg ethers, and cyclic ethers. Aromatic hydrocarbons used as crystallization solvents include C₄-C₆ alkyl aromatic solvents which may include substituted aromatics. Examples of aromatic hydrocarbons include, but are not limited to toluene, benzene, and the like. The term “C₅-C₉ hydrocarbons” refer to C₅-C₉ alkyl solvents which may be substituted, branched or unbranched alkyl. Such hydrocarbon solvents include, but are not limited to straight or branched heptane, octane, pentane, and the like. The term “C₃-C₉ ketones” refers to straight or branched ketones which may optionally be substituted. The term “C₃-C₉ esters” refers to straight or branched esters which may optionally be substituted. The term “ethers” refer to lower alkyl (C₂-C₈) alkyl ethers which may be straight, branched or substituted. The term ether shall include but is not limited to, for example, t-butyl methylether, and the like. The term “cyclic ether” includes C₅-C₇ cyclic ether which may be optionally substituted. Optionally, the ether solvent is dry. Optionally, such dry solvent shall contain less than about 1% water. Urea and urea derivatives can also be used as crystallization solvents.

Crystallization solvents are selected on the basis that a compound of formula (I) must be at least partially soluble in the solvent selected, and the solvent selected must not form a solvate with the compound of formula (I). Optionally, the solvate dissolves in the solvent before the crystallization process is begun.

Crystallization solvents used in making LSN 487355 and LSN 509207 forms include 1,4-dioxane (dioxane), 1,2-dichloroethane, dimethoxyethane, glycols including ethylene glycol and diethylene glycol, dimethyl ether, tetrahydrofuran (THF), isopropyl acetate, diisopropyl ether, hexane, heptane, cyclohexane, toluene or xylene, alcohols such as methanol, ethanol, 1-propanol, 2-propanol, 1-butanol, 2-butanol, and tert-butanol, ketones such as methyl ethyl ketone or isobutyl methyl ketone, amides such as dimethylformamide, dimethylacetamide or N-methylpyrrolidone, pyridine, DMSO, xylene, urea or urea derivatives, acetic acid, and mixtures thereof. For example, crystallization solvents comprise one or more solvents selected from the group consisting of ethanol, methanol, isopropanol, ethyl acetate, acetone, 1,2-dichloroethane, THF, acetonitrile, nitromethane, 1,4-dioxane, pyridine, DMSO, and acetic acid.

“Glycols” include, but are not limited to, ethylene glycol, 1,3-propane diol, propylene glycol, glycol ethers including dipropylene glycolpropyl ether, dipropylene glycolbutyl ether, and diethylene glycol butyl ether.

The term “patient” is used throughout the specification to describe an animal, such as a human, to whom treatment, including prophylactic treatment, with the compositions according to the present invention is provided. For treatment of those infections, conditions or disease states which are specific for a specific animal such as a human patient, the term patient refers to that specific animal.

The term “neoplasia” is used to describe the pathological process that results in the formation and growth of a neoplasm, i.e., an abnormal tissue that grows by cellular proliferation more rapidly than normal tissue and continues to grow after the stimuli that initated the new growth cease. Neoplasia exhibits partial or complete lack of structural organization and functional coordination with the normal tissue, and usually forms a distinct mass of tissue which may be benign (benign tumor) or malignant (carcinoma). The term “cancer” is used as a general term to describe any of various types of malignant neoplasms, most of which invade surrounding tissues, may metastasize to several sites and are likely to recur after attempted removal and to cause death of the patient unless adequately treated. As used herein, the term cancer is subsumed under the term neoplasia.

The term “inhibitory effective concentration” or “inhibitory effective amount” is used throughout the specification to describe concentrations or amounts of compositions according to the present invention which substantially or significantly inhibit the growth or replication of susceptible neoplasias.

The terms “an effective amount”, “therapeutic effective amount”, or “therapeutically effective amount” shall mean an amount or concentration of a composition according to the present invention which is effective within the context of its administration or use, including, for example, the treatment of neoplasias. Thus, the term “effective amount” is used throughout the specification to describe concentrations or amounts of compounds according to the present invention which may be used to produce a favorable change in the disease or condition treated, whether that change is a remission, a decrease in growth or size of cancer or a tumor or other favorable physiological result.

A further aspect of the present invention relates to the treatment of neoplasia, including cancer, comprising administering to a patient in need thereof an effective amount of a compound as described hereinabove, optionally in combination with a pharmaceutically acceptable additive, carrier or excipient. The present invention also relates to methods for inhibiting the growth of neoplasia, including a malignant tumor or cancer comprising exposing the neoplasia to an inhibitory or therapeutically effective amount or concentration of at least one of the disclosed compounds. This method may be used therapeutically, in the treatment of neoplasia, including cancer or in comparison tests such as assays for determining the activities of related analogues as well as for determining the susceptibility of a patient's cancer to one or more of the compounds according to the present invention. Primary utility resides in the treatment of neoplasia, including cancer, especially including lung cancer, breast cancer and prostate cancer, among others.

A therapeutic aspect according to the present invention relates to methods for treating neoplasia, including benign and malignant tumors and cancer in animal or human patients, and in specific embodiments, cancers which have developed drug resistance, including, for example, multiple drug resistant breast cancer comprising administering therapeutically effective amounts or concentrations of one or more of the compounds according to the present invention to inhibit the growth or spread of or to actually shrink the neoplasia in the animal or human patient being treated.

Cancers which may be treated using compositions according to the present invention include, for example, stomach, colon, rectal, liver, pancreatic, lung, breast, cervix uteri, corpus uteri, ovary, prostate, testis, bladder, renal, brain/cns, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney and lymphoma, among others. Compounds according to the present invention are particularly useful in the treatment of lung cancer, breast cancer and prostate cancer.

In the present methods, in certain embodiments, it has been found advantageous to coadminister at least one additional anti-neoplastia agent for the treatment of neoplasia, including cancer. In these aspects according to the present invention, an effective amount of one or more of the compositions according to the present invention is co-administered along with an effective amount of at least one additional anti-neoplastia/anti-cancer agent such as, for example a nucleoside.

Pharmaceutical Compositions and Dosage Forms

Pharmaceutical dosage forms of the invention can be administered orally, parenterally, by inhalation spray, topically, rectally, nasally, buccally, vaginally or via an implanted reservoir. Oral and parenteral pharmaceutical compositions and dosage forms are exemplary dosage forms. For example, the oral dosage form is a solid dosage form, such as a tablet, a caplet, a hard gelatin capsule, a starch capsule, a hydroxypropyl methylcellulose (HPMC) capsule, or a soft elastic gelatin capsule. Other dosage forms include an intradermal dosage form, an intramuscular dosage form, a subcutaneous dosage form, and an intravenous dosage form.

Pharmaceutical compositions and dosage forms of the invention comprise an active ingredient as disclosed herein, e.g., a form such as a co-crystal, solvate, or salt form of LSN 487355 or LSN 509207. Pharmaceutical compositions and unit dosage forms of the invention typically also comprise one or more pharmaceutically acceptable excipients or diluents. In one embodiment, the pharmaceutical compositions and unit dosage forms of the invention typically also comprise one or more pharmaceutically acceptable excipients or diluents, wherein at least one of the pharmaceutically acceptable excipients or diluents is an antioxidant.

Pharmaceutical unit dosage forms of this invention are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules, starch capsules, hydroxypropyl methylcellulose (HPMC) capsules, and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease or disorder may contain larger amounts of the active ingredient than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990) or Remington: The Science and Practice of Pharmacy, 19th ed., Mack Publishing, Easton Pa. (1995).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. In addition, pharmaceutical compositions or dosage forms may contain one or more compounds that reduce or alter the rate by which the active ingredient will decompose. Such compounds, which are referred to herein as “stabilizers”, include, but are not limited to, antioxidants, pH buffers, or salt buffers.

One or more antioxidants can be used in pharmaceutical compositions and dosage forms to deter radical oxidation of the active ingredient, wherein such antioxidants include, but are not limited to, ascorbic acid, phenolic antioxidants including, but not limited to, butylated hydroxyanisole (BHA) and propyl gallate, and chelators including, but not limited to citrate, EDTA, and DTPA. For example, in cases where radical oxidation of the active ingredient is known to occur, a combination of phenolic antioxidants and chelators can be used.

Like the amounts and types of excipients, the amounts and specific type of active ingredient in a dosage form may differ depending on factors such as, but not limited to, the route by which it is to be administered to patients. However, typical dosage forms of the invention comprise a form of a compound of formula (I), such as a form of LSN 487355 or LSN 509207, in an amount of from about 10 mg to about 1000 mg, from about 25 mg to about 500 mg, from about 40 mg to 400 mg, or from about 50 mg to about 200 mg.

Oral Dosage Forms

Pharmaceutical compositions of the invention that are suitable for oral administration can be presented as discrete dosage forms, such as, but not limited to, tablets (including without limitation scored or coated tablets), pills, caplets, capsules (including without limitation hard gelatin capsules, starch capsules, HPMC capsules, and soft elastic gelatin capsules), chewable tablets, powder packets, sachets, troches, wafers, aerosol sprays, or liquids, such as but not limited to, syrups, elixirs, solutions or suspensions in a non-aqueous liquid, an oil-in-water emulsion, or a water-in-oil emulsion. Such compositions contain a predetermined amount of the active ingredient, and may be prepared by methods of pharmacy well known to those skilled in the art. See generally, Remington's Pharmaceutical Sciences, 18th ed., Mack Publishing, Easton Pa. (1990) or Remington: The Science and Practice of Pharmacy, 19th ed., Mack Publishing, Easton Pa. (1995).

Typical oral dosage forms of the invention are prepared by combining the active ingredient in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, stabilizers, and disintegrating agents.

Due to their ease of administration, tablets, caplets, and capsules (such as hard gelatin, HPMC, or starch capsules) represent the most advantageous solid oral dosage unit forms, in which case solid pharmaceutical excipients are used. If desired, tablets or caplets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the invention include, but are not limited to, binders, stabilizers, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants can be used in the pharmaceutical compositions and dosage forms to provide tablets or caplets that disintegrate when exposed to an aqueous environment. Tablets or caplets that contain too much disintegrant may disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the invention. The amount of disintegrant used varies based upon the type of formulation and mode of administration, and is readily discernible to those of ordinary skill in the art. Typical pharmaceutical compositions comprise from about 0.5 to about 15 weight percent of disintegrant, for example, from about 1 to about 5 weight percent of disintegrant.

Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Antioxidants can be used in the pharmaceutical compositions and dosage forms to deter degradation or radical oxidation of the active ingredient. Examples of suitable antioxidants include, but are not limited to, ascorbic acid, phenolic antioxidants including, but not limited to, butylated hydroxyanisole (BHA) and propyl gallate, and chelators including, but not limited to, citrate, EDTA, and DTPA, or combinations thereof.

Lubricants that can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Other oral dosage forms for pharmaceutical compositions of the invention are soft elastic gelatin capsules. Soft elastic gelatin capsule unit dosage forms can be made using conventional methods well known in the art. See, e.g., Ebert, Pharm. Tech, 1(5):44-50 (1977). In general, soft elastic gelatin capsules (also known as “soft gels”) have an elastic or soft, globular or oval shaped gelatin shell that is typically a bit thicker than that of hard gelatin capsules, wherein a plasticizing agent, e.g., glycerin, sorbitol, or a similar polyol, is added to a gelatin. The type of gelatin, as well as the amounts of plasticizer and water, can be used to vary the hardness of the capsule shell. The soft gelatin shells may contain a preservative, such as methyl- and propylparabens and sorbic acid, to prevent the growth of fungi. The active ingredient may be dissolved or suspended in a liquid vehicle or carrier, such as vegetable or mineral oils, glycols, such as polyethylene glycol and propylene glycol, triglycerides, surfactants, such as polysorbates, or a combination thereof.

Controlled and Delayed Release Dosage Forms

Pharmaceutically acceptable forms of LSN 487355 or LSN 509207 can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the forms and compositions of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed forms of LSN 487355 or LSN 509207 and thus effect controlled delivery of the drug. Examples of specific anion exchangers include, but are not limited to, Duolite® A568 and Duolite® AP143 (Rohm & Haas, Spring House, Pa. USA).

One embodiment of the invention encompasses a unit dosage form which comprises a pharmaceutically acceptable form of LSN 487355 or LSN 509207 (e.g., a salt or a solvate), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B 1; 6,368,626 B 1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder (e.g., a LSN 487355 or LSN 509207 form) into a hard tablet, coating the tablet with cellulose derivatives to form a semi-permeable membrane, and then drilling an orifice in the coating (e.g., with a laser) (Kim, Cherng-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a form of LSN 487355 or LSN 509207. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a form of LSN 487355 or LSN 509207. See U.S. Pat. No. 6,342,249, the entirety of which is incorporated herein by reference.

An example of a delayed-release dosage form that also functions as a time controlled-release dosage form is described in U.S. Pat. No. 5,366,738, herein incorporated by reference in its entirety. The controlled-release drug delivery device described in U.S. Pat. No. 5,366,738 is known as a gel extrusion module (GEM) delivery device. The GEM device is a drug delivery device for the controlled in situ production and release of a dispersion containing a beneficial agent such as a pharmaceutical drug comprising:
(a) a compressed core prepared from an admixture comprising:
   (i) a therapeutically effective amount of the beneficial agent; and
   (ii) a polymer which upon hydration forms gelatinous microscopic particles; and
(b) a water insoluble, water impermeable polymeric coating comprising a polymer and a plasticizer, which surrounds and adheres to the core, the coating having a plurality of formed apertures exposing between about 1 and about 75% of the core surface; and wherein the release rate of the beneficial agent from the device is a function of the number and size of the apertures.

In the GEM device, the polymer inside the compressed core is selected from materials such as sodium polyacrylate, carboxypolymethylenes and the pharmaceutically acceptable salts thereof such as a sodium salt, wherein the carboxypolymethylenes are prepared from acrylic acid crosslinked with allylethers of sucrose or pentaerythritol, and, for example, it is selected from carboxypolymethylenes prepared from acrylic acid crosslinked with allylethers of sucrose or pentaerythritol, and the pharmaceutically acceptable salts thereof. Often CARBOPOL® 974P and pharmaceutically acceptable salts thereof, particularly the sodium salt, is used as the polymer inside the compressed core. In addition, the compressed core may also contain one or more polymer hydration modulating agents, anti-oxidants, lubricants, fillers and excipients. An optional subcoating may be applied to the compressed core prior to application of the water insoluble coating as an aid in the manufacturing process. The subcoating may be comprised of, for example, hydroxypropyl cellulose and hydroxypropylmethylcellulose. Additional coatings may be applied for aesthetic or functional purposes.

The water insoluble, water impermeable polymeric coating is comprised of, for example, (1) a polymer selected from polyvinyl chloride, cellulose acetate, cellulose acetate butyrate, ethylcellulose and combinations of these polymers; and (2) a plasticizer selected from diethylphthalate, dibutylsebacate and triethylcitrate. For example, the polymeric coating is comprised of cellulose acetate butyrate and triethyl citrate. The GEM device does not function as an osmotic drug delivery device, hence the release function of the device depends on passage of fluids from the external environment of the body to the internal environment of the compressed core through the formed apertures. It is intended that the terms “water insoluble, water impermeable” used to describe the polymeric coating define a coating which is essentially water insoluble and water impermeable, meaning that the polymeric coating allows minimal to no passage of water through the coating from the external environment of the body to the internal environment of the compressed core, except for the fluid passage that occurs through the drilled apertures, during the period of time the drug is being released from the GEM device in the body. Any minimal amount of water that does pass through the water insoluble, water impermeable polymeric coating is insubstantial and does not significantly contribute to the function of the GEM device, i.e. the release rate of the drug through the apertures. Rather the release rate of soluble forms of the present invention from the GEM device is primarily a function of the number and size of the apertures on the device.

For an elegant, aesthetically pleasing final product, an outer finish coat may finally be applied to the GEM delivery device containing colorants, waxes, and the like. The GEM device can also be enterically coated, either before or after the application of additional finish coatings. Even without enteric coating, extrusion of the polymer which carries soluble forms of the present invention out from inside the compressed core of the GEM device does not occur to a substantial extent in the acidic pH of the stomach, therefore substantial release of soluble forms of the present invention should not occur in the stomach. Further details and examples of the GEM delivery device are described in U.S. Pat. No. 5,366,738.

Topical Dosage Forms

Topical dosage forms of the invention include, but are not limited to, creams, lotions, ointments, gels, shampoos, sprays, aerosols, solutions, emulsions, and other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity, optionally, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, which can be in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th ed., Mack Publishing, Easton, Pa. (1990).

Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are, optionally, sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. The solutions are, optionally, isotonic and have a physiological pH.

Compounds that increase the solubility the active ingredient(s) disclosed herein can also be incorporated into the parenteral dosage forms of the invention.

Transdermal and Mucosal Dosage Forms

Transdermal and mucosal dosage forms of the invention include, but are not limited to, ophthalmic solutions, patches, sprays, aerosols, creams, lotions, suppositories, ointments, gels, solutions, emulsions, suspensions, or other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). Dosage forms suitable for treating mucosal tissues within the oral cavity can be formulated as mouthwashes, as oral gels, or as buccal patches. Further, transdermal dosage forms include “reservoir type” or “matrix type” patches, which can be applied to the skin and worn for a specific period of time to permit the penetration of a desired amount of active ingredient.

Suitable excipients (e.g., carriers and diluents) and other materials that can be used to provide transdermal and mucosal dosage forms encompassed by this invention are well known to those skilled in the pharmaceutical arts, and depend on the particular tissue or organ to which a given pharmaceutical composition or dosage form will be applied. With that fact in mind, typical excipients include, but are not limited to Labrasol, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with active ingredients of the invention. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, solvates, polymorphs, or co-crystals of the active ingredient can be used to further adjust the properties of the resulting composition.

In one embodiment of the invention, an active ingredient comprising a form of LSN 487355 or LSN 509207 is administered orally as needed in an amount of from about 10 mg to about 1000 mg, from about 25 mg to about 500 mg, from about 40 mg to about 400 mg, or from about 50 mg to about 200 mg. The dosage amounts can be administered in single or divided doses. The dosage amounts and frequencies provided above are encompassed by the term “inhibitory effective amount” as used herein.

The suitability of a particular route of administration employed for a particular active ingredient will depend on the active ingredient itself (e.g., whether it can be administered orally without decomposing prior to entering the blood stream) and the disease or disorder to be treated or prevented.

Preparation of Active Ingredient and Forms Thereof

Compounds of formula (I) can be made using various methods known to those skilled in the art, including those disclosed in U.S. Pat. No. 6,673,809. LSN 487355 or LSN 509207 can be made by such techniques.

Forms including salts, co-crystals, or solvates of compounds of formula (I), such as LSN 487355 or LSN 509207, may be prepared by reacting the compound with an appropriate acid, such as an organic or inorganic acid, including without limitation, oxalic acid, succinic acid, malic acid, hydrochloric acid, sulfuric acid, fumaric acid, phosphoric acid, tartaric acid, maleic acid, malonic acid, adipic acid, and benzenesulfonic acid. For example, the process for forming a salt and co-crystal can be carried out in a crystallization solvent in which both reactants (i.e., the free base compound and acid) are sufficiently soluble.

In one method, in order to achieve crystallization or precipitation, a crystallization solvent is used in which the resulting form, e.g., salt or co-crystal, is only slightly soluble or not soluble. Alternatively, a crystallization solvent is used in which the desired salt and co-crystal is very soluble, and an anti-solvent (or a crystallization solvent in which the resulting salt is poorly soluble) is added to the solution. Other variants for salt formation or crystallization include concentrating the salt and co-crystal solution (e.g., by heating, under reduced pressure if necessary, or by slowly evaporating the solvent, for example, at room temperature), or seeding with the addition of seed crystals, or setting up water activity required for hydrate formation.

Table I presents data reflecting a variety of physical properties of LSN 487355 and LSN 509207.

TABLE I
Physical properties of the free base forms of LSN 487355 and
LSN 509207.

| | | |
|---|---|---|
| Serial Number | LSN 487355 | LSN 509207 |
| Microscopy | Birefringent | Birefringent |
| Physical state (PXRD) | Crystalline | Crystalline |
| Endothermic transition (DSC) | 206.6 degrees C. | 212.7 degrees C. |
| Glass Transition (Tg) | 96 degrees C. | 103 degrees C. |
| Volatiles (TGA) | 0.15% | 0.9% |
| Purity (HPLC) | 99.8% | 97% |
| Hygroscopicity (VTI) | Non-hygroscopic | Non-hygroscopic |

Table II presents data reflecting absorption behavior and permeability parameters of LSN 487355 and LSN 509207.

TABLE II
Absorption and permeability parameters of the free base
forms of LSN 487355 and LSN 509207.

| Serial Number | LSN 487355 | LSN 509207 |
|---|---|---|
| Molecular Weight | 468.9 | 483.9 |
| clog P | 3.97 | 3.57 |
| pKa | 3.77 | — |
| Polar surface area | 85 angstroms² | 97 angstroms² |
| cPeff (Winiwarter 2b) | 1.7 × 10⁻⁴ cm/sec | 0.7 × 10⁻⁴ cm/sec |
| Papp (Caco-2) | 7.9 × 10⁻⁶ cm/sec | 7.4 × 10⁻⁶ cm/sec |
| Permeability class | Borderline high | Borderline high |
| B-A/A-B ratio | 2.3 | 2.7 |

The solubility of LSN 487355 in a variety of vehicles is illustrated in Table III.

TABLE III
Solubility of LSN 487355 free base in various solvents/solubilizing
mixtures

| Solvent/Solubilizing Mixture | Solubility (mg/mL) |
|---|---|
| NMP | >563 |
| DMA | 208 < S < 416 |
| Capmul ® PG8 | 12 < S < 15 |
| Labrasol | 4 < S < 5 |
| PEG 300 | >2 |
| Labrafac Hydro WL 219 | >2 |
| Glycofurol | >2 |
| Phosphal 53 MCT | >2 |
| Phosphal 50 PG | >2 |
| Soybean oil | <2 |
| Safflower oil | <2 |
| Olive oil | <2 |
| Labrafil M1944CS | <2 |
| Labrafil M2125CS | <2 |
| Labrafac Lipophile WL 1349 | <2 |
| Ethyl Oleate | <2 |
| Propylene Glycol | <2 |
| 4% DMSO** | 0.002 |
| 10% DMSO** | 0.004 |
| DMSO | ≧51* |
| Ethanol | 1.4 |
| 5% N-methyl-2-pyrrolidone** | 0.004 |
| 10% N-methyl-2-pyrrolidone** | 0.03 |
| N-methyl-2-pyrrolidone | ≧52* |
| Ethyl acetate | 0.003 |

*Solubility measured visually at about 50 mg/mL; all compound dissolved
**Compound dissolved in solvent first, then deionized water added

Having ascertained the physical and pharmacokinetic properties of LSN 487355 and LSN 509207 presented herein, a series of experiments were undertaken to make novel pharmaceutically acceptable forms of LSN 487355 and LSN 509207. The experiments described in the examples provided hereinafter are illustrative.

Novel lipid soluble forms of the invention also include, but are not limited to dimethyl sulfoxide (DMSO), methyl tert-butyl ether (MTBE), formamide, acetonitrile, nitromethane, methylene chloride, acetic acid, pyridine, 1,4-dioxane, tetrahydrofuran (THF), nitromethane, tris base, and isopropyl acetate (IPA) solvates of a pharmaceutically acceptable salt or the freebase or a polymorph of the freebase of a compound of LSN 487355 and LSN 509207. Soluble crystalline forms of the invention include:
1. a dimethyl sulfoxide, methyl tert-butyl ether, or formamide solvate of LSN 487355;
2. a phosphate salt obtained by the crystallization of LSN 487355 in a crystallization solvent comprising phosphoric acid and acetonitrile, wherein the phosphate salt of LSN 487355 is formed;
3. a nitromethane solvate of a phosphate salt of LSN 487355 formed by the crystallization of the phosphate salt of LSN 487355 in a crystallization solvent comprising nitromethane and THF;
4. a nitromethane solvate of an oxalate salt of LSN 487355 formed by the crystallization of the oxalate salt of LSN 487355 in a crystallization solvent comprising nitromethane;
5. a dinitromethane solvate of an oxalate salt of LSN 487355 formed by the crystallization of the oxalate salt of LSN 487355 in a crystallization solvent comprising dinitromethane;
6. a nitrate salt of LSN 487355 formed by the crystallization of LSN 487355 in a crystallization solvent comprising nitric acid, toluene and isopropyl acetate, wherein the nitrate salt is formed;
7. LSN 487355 methanesulfonic acid co-crystal or salt form obtained by the crystallization of LSN 487355 in a crystallization solvent comprising methanesulfonic acid and either methanol alone, methanol and water, or methanol and hydrogen bromide;
8. LSN 487355:fumaric acid co-crystal formed by the crystallization of LSN 487355 free base in a crystallization solvent comprising fumaric acid and nitromethane;
9. LSN 509207 Form II was obtained by the crystallization of LSN 509207 in a crystallization solvent comprising nitromethane and isopropyl acetate in the presence of succinic acid;
10. LSN 509207 Form III was obtained by the crystallization of LSN 509207 in a crystallization solvent selected from the group consisting of (i) 1,2-dichloroethane and tris base; (ii) acetone; (iii) 1,2-dichloroethane and either imidazole or saccharin; and (iv) 1,2-dichloroethane;
11. LSN 509207 Form IV was obtained by the crystallization of LSN 509207 in a crystallization solvent selected from the group consisting of (i) acetonitrile; (ii) nitromethane, isopropanol, and nicotinamide; and (iii) acetonitrile, isopropanol, and caffeine;
12. LSN 509207 Form V was obtained by the crystallization of LSN 509207 in a crystallization solvent selected from the group consisting of (i) n-heptane, 1,2-dichloroethane and acetaminophen; and (ii) water, 1,2-dichloroethane and citric acid;
13. LSN 509207 solvates formed by the crystallization of LSN 509207 in a crystallization solvent selected from the group consisting of methylene chloride, acetone, acetonitrile, nitromethane, THF, 1,4-dioxane, pyridine, and acetic acid; and
14. LSN 509207:1-hydroxy-2-naphthoic acid co-crystals formed by the crystallization of LSN 509207 in a crystallization solvent comprising 1,2-dichloroethane.

Liquid pharmaceutical formulations of the invention comprise: a pharmaceutically acceptable salt, co-crystal, solvate, or polymorph of LSN 487355 or LSN 509207 and an excipient system comprising Labrasol, wherein the excipient system comprises approximately 25% to 75% by weight of the formulation. The lipid solubility of LSN 487355 or LSN 509207 in the excipient system is approximately 10 mg/ml to approximately 150 mg/ml, and wherein the formulation can be administered orally or parenterally.

A liquid pharmaceutical formulation of the invention comprises a phosphate salt of LSN 487355 and an excipient system comprising either:
(1) Labrasol, wherein the excipient system comprises approximately 25% to 75% by weight of the formulation, the lipid solubility of LSN 487355 in the excipient system is approximately 80 mg/ml to approximately 120 mg/ml; or
(2) approximately equal amounts on a weight percentage basis of Labrasol and a PEG, wherein the excipient system comprises approximately 25% to 75% by weight of the formulation, the solubility of LSN 487355 in the excipient system is approximately 80 mg/ml to approximately 120 mg/ml, and wherein the formulation can be administered orally or parenterally.

The invention is described further in the following examples, which are illustrative and in no way limiting.

### Analytical Equipment and Procedures

### Thermogravimetric Analysis

Thermogravimetic analysis of each sample was performed using a Q500 Thermogravimetric Analyzer (TA Instruments, New Castle, Del., U.S.A.), which uses as its control software Advantage for QW-Series, version 1.0.0.78, Thermal Advantage Release 2.0 (© 2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©1991-2001 TA Instruments-Water LLC).

For all of the experiments, the basic procedure for performing thermogravimetric analysis comprised transferring an aliquot of a sample into a platinum sample pan (Pan part # 952019.906; (TA Instruments, New Castle, Del. USA)). The pan was placed on the loading platform and was then automatically loaded into the Q500 Thermogravimetric Analyzer using the control software. Thermograms were obtained by individually heating the sample at 110° C./minute across a temperature range (generally from 25° C. to 300° C.) under flowing dry nitrogen (compressed nitrogen, grade 4.8 (BOC Gases, Murray Hill, N.J. USA)), with a sample purge flow rate of 60 mL/minute and a balance purge flow rate of 40 mL/minute. Thermal transitions (e.g., weight changes) were viewed and analyzed using the analysis software provided with the instrument.

### Differential Scanning Calorimetry

DSC analysis of each sample was performed using a Q1000 Differential Scanning Calorimeter (TA Instruments, New Castle, Del., U.S.A.), which uses Advantage for QW-Series, version 1.0.0.78, Thermal Advantage Release 2.0 (© 2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL-version 1.0.0.78 build 78.1. In addition, the analysis software used was Universal Analysis 2000 for Windows 95/95/2000/NT, version 3.1E; Build 3.1.0.40 (©2001 TA Instruments-Water LLC).

For all of the DSC analyses, an aliquot of a sample was weighed into an aluminum sample pan (Pan part # 900786.091; lid part # 900779.901 (TA Instruments, New Castle Del. USA)). The sample pan was sealed either by crimping for dry samples or press fitting for wet samples (such as hydrated or solvated samples). The sample pan was loaded into the Q1000 Differential Scanning Calorimeter, which is equipped with an autosampler, and a thermogram was obtained by individually heating the same using the control software at a rate of 10° C./minute from Tₘᵢₙ (typically 30° C.) to Tₘₐₓ (typically 300° C.) using an empty aluminum pan as a reference. Dry nitrogen (compressed nitrogen, grade 4.8 (BOC Gases, Murray Hill, N.J. USA)) was used as a sample purge gas and was set at a flow rate of 50 mL/minute. Thermal transitions were viewed and analyzed using the analysis software provided with the instrument.

### Powder X-Ray Diffraction

All X-ray powder diffraction patters were obtained using a D/Max Rapid X-ray Diffractometer (Rigaku/MSC, The Woodlands, Tex., U.S.A.) equipped with a copper source (Cu/K_{α}1.5406Å), manual x-y stage, and 0.3 mm collimator. A sample was loaded into a 0.3 mm quartz capillary tube (Charles Supper Company, Natick, Mass., U.S.A.) by sectioning off the closed end of the tube and tapping the small, open end of the capillary tube into a bed of the powdered sample or into the sediment of a slurried sample. The precipitate can be amorphous or crystalline. The loaded capillary tube was mounted in a holder that was placed and fitted into the x-y stage. A diffractogram was acquired using control software (RINT Rapid Control Software, Rigaku Rapid/XRD, version 1.0.0 (© 1999 Rigaku Co.)) under ambient conditions at a power setting of 46 kV at 40 mA in transmission mode, while oscillating about the omega-axis from 0-5 degrees at 1 degree/second, and spinning about the phi-axis over 360 degrees at 2 degrees/second. The exposure time was 15 minutes unless otherwise specified.

The diffractogram obtained was integrated of 2-theta from 2-60 degrees and chi (1 segment) from 0-36 degrees at a step size of 0.02 degrees using the cyllnt utility in the RINT Rapid display software (RINT Rapid display software, version 1.18 (Rigaku/MSC)) provided by Rigaku with the instrument. The dark counts value was set to 8 as per the system calibration by Rigaku. No normalization or omega, chi or phi offsets were used for the integration.

Exemplification

### EXAMPLE 1

LSN 487355 Phosphate Salt

The phosphate salt of LSN 487355 was obtained by adding 1.05 equivalents of phosphoric acid dissolved in acetonitrile to 25 mg LSN 487355. A vortex was used to mix the above materials followed by heating in a hot water bath until the contents were clear. The mixture was then cooled to 5 degrees C. for several hours during which time crystallization of the phosphate salt occurred. Excess supernatant was removed with a syringe, and the remaining sample was dried under nitrogen. The phosphate salt of LSN 487355 was also obtained from mixtures of nitromethane and tetrahydrofuran (THF).

Another method for the synthesis of the phosphate salt was completed by adding 300 mg of LSN 487355 with 12 mL acetonitrile containing 1.05 molar equivalent of phosphoric acid in an amber glass vial. The vial was then capped and sealed with parafilm. Heating in hot water with vortexing and sonication were completed in an alternating fashion until the solution was clear. The solution was seeded with a small amount of LSN 487355 phosphate salt, and equilibrated at room temperature for several hours, then at 5 degrees C. for several more hours. The salt crystallized over the course of a few hours. Excess supernatant was decanted into a vial and removed with a pipet. The resultant crystals were washed with cold acetonitrile and again decanted with pipet/syringe, then dryed under flowing nitrogen. Additional material can be recovered from the supernatant by evaporating under nitrogen until a small amount of solvent remains, equilibrating the remaining sample for several hours, and isolating powder as above.

Dynamic vapor sorption of the phosphate salt showed a loss of crystallinity when equilibrated under flowing nitrogen (0% RH), displayed a significant moisture sorption isotherm, and began to deliquesce above about 80-90% RH.

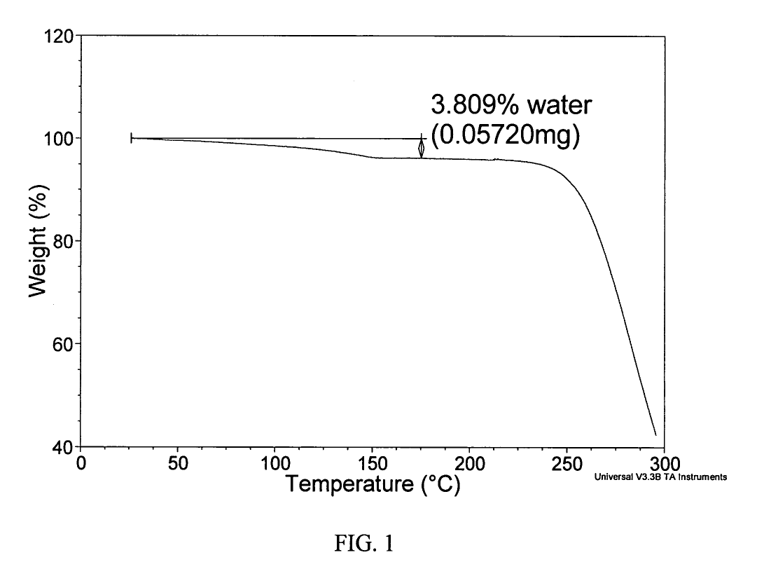

TGA analysis of the LSN 487355 phosphate salt showed about a 3.8 percent weight loss between room temperature and about 175 degrees C. with a heating rate of 10 degrees C./minute (FIG. 1).

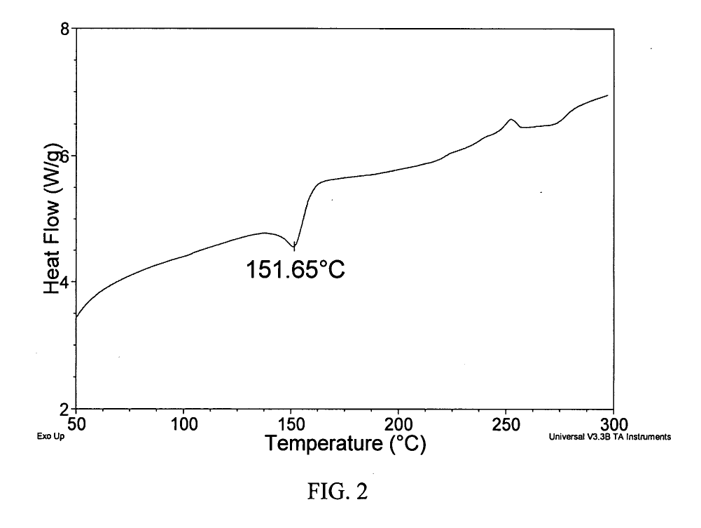

DSC analysis of the LSN 487355 phosphate salt showed an endothermic transition at about 152 degrees C. with a heating rate of 10 degrees C./minute (FIG. 2).

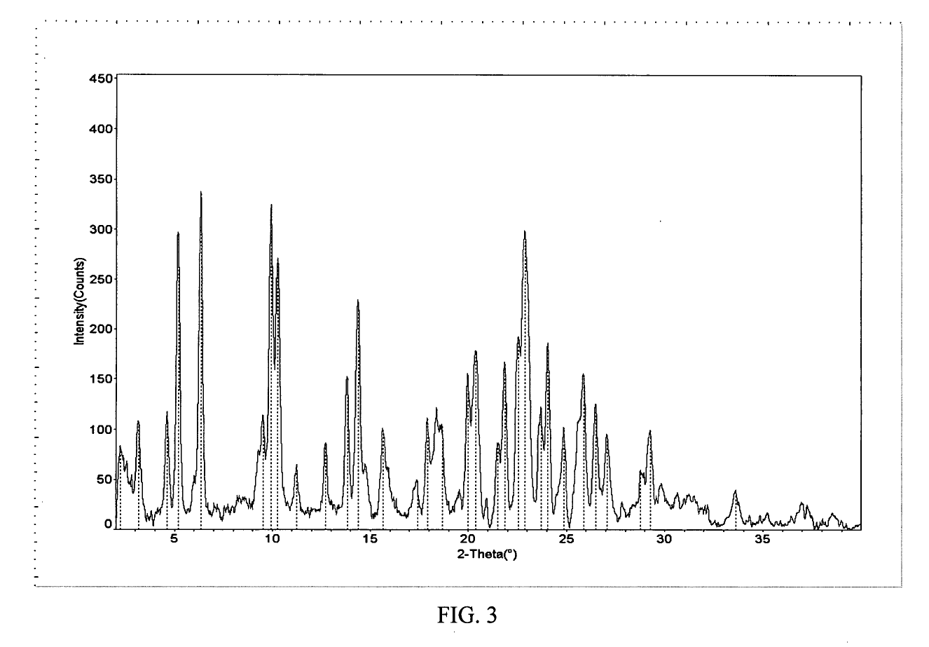

PXRD analysis of the LSN 487355 phosphate salt was completed. The LSN 487355 phosphate salt can be characterized by any one, any two, any three, any four, any five, or any six or more of the peaks in FIG. 3 including, but not limited to, 5.19, 6.35, 9.95, 10.27, 14.39, 19.99, 20.39, 21.87, 22.55, 22.89, and 24.05 degrees 2-theta.

### EXAMPLE 2

LSN 487355 Phosphate Salt Nitromethane Solvate

50 mg of the LSN 487355 phosphate salt nitromethane solvate was obtained by dissolving 1.05 equivalents of phosphoric acid in a 1:1 (by volume) THF:nitromethane mixture. Additional nitromethane was added while vortexing and heating in a hot water bath until clear. The solvate precipitated immediately upon cooling. The sample was allowed to sit overnight followed by removal of the excess supernatant with a syringe. The sample was dried under nitrogen.

TGA analysis of the LSN 487355 phosphate salt nitromethane solvate showed about a 6.7 percent weight loss between room temperature and about 150 degrees C. with a heating rate of 10 degrees C./minute.

DSC analysis of the LSN 487355 phosphate salt nitromethane solvate showed an endothermic transition at about 151.5 degrees C. with a heating rate of 10 degrees C./minute.

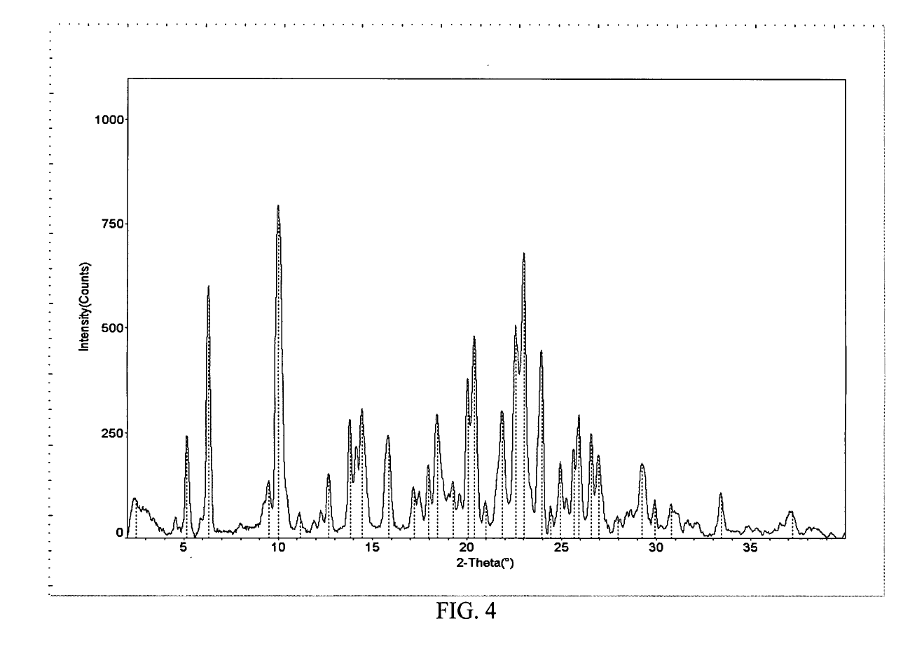

PXRD analysis of the LSN 487355 phosphate salt nitromethane solvate was completed. The LSN 487355 phosphate salt nitromethane solvate can be characterized by any one, any two, any three, any four, any five, or any six or more of the peaks in FIG. 4 including, but not limited to, 5.17, 6.33, 9.99, 13.82, 14.47, 15.85, 18.43, 20.05, 20.39, 21.87, 22.59, 23.03, 23.95 and 25.93 degrees 2-theta.

### EXAMPLE 3

LSN 487355 Oxalate Salt Nitromethane Solvate

The LSN 487355 oxalate salt nitromethane solvate was obtained using a 1:1 (molar) mixture of LSN 487355 and oxalic acid. LSN 487355 was dissolved in nitromethane prior to combination with an oxalic acid solution in THF. A vortex was used to mix the above materials followed by heating in a hot water bath until the contents were clear. The mixture was then cooled to 5 degrees C. for several hours during which time crystallization of the LSN 487355 oxalate salt nitromethane solvate occurred. Excess supernatant was removed with a syringe, and the remaining sample was dried under nitrogen.

TGA analysis of the LSN 487355 oxalate salt nitromethane solvate showed about a 24.6 percent weight loss between room temperature and about 150 degrees C. with a heating rate of 10 degrees C./minute.

DSC analysis of the LSN 487355 oxalate salt nitromethane solvate showed endothermic transitions at about 54.9, 85.7, and 132.6 degrees C. with a heating rate of 10 degrees C./minute.

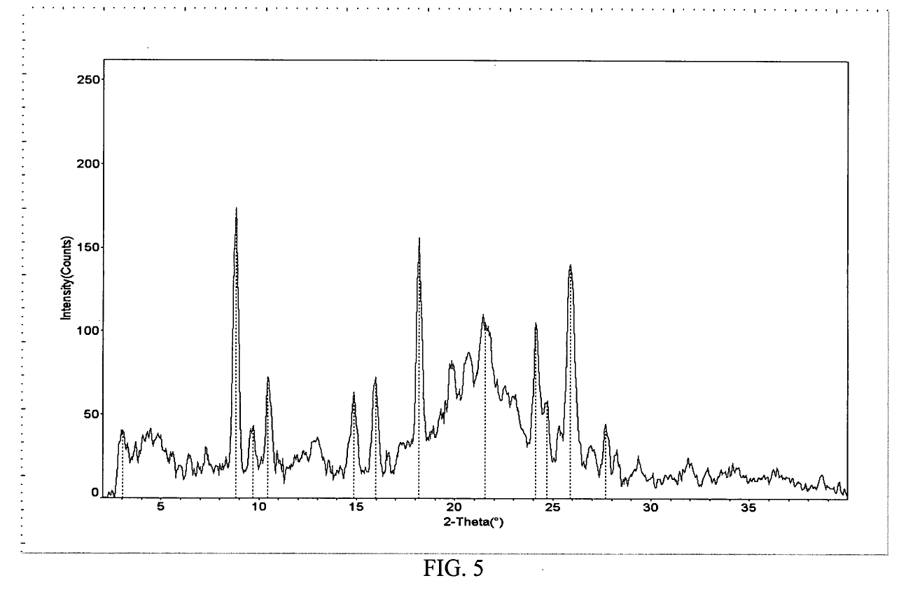

PXRD analysis of the LSN 487355 oxalate salt nitromethane solvate was completed. The LSN 487355 oxalate salt nitromethane solvate can be characterized by any one, any two, any three, any four, any five, or any six or more of the peaks in FIG. 5 including, but not limited to, 8.82, 10.45, 14.85, 15.97, 18.18, 21.55, 24.12, and 25.87 degrees 2-theta.

### EXAMPLE 4

LSN 487355 Oxalate Salt Dinitromethane Solvate

The LSN 487355 oxalate salt dinitromethane solvate was obtained using a 1:1 (molar) mixture of LSN 487355 and oxalic acid. LSN 487355 was dissolved in dinitromethane prior to combination with an oxalic acid solution in THF. A vortex was used to mix the above materials followed by heating in a hot water bath until the contents were clear. The mixture was then cooled to 5 degrees C. for several hours during which time crystallization of the LSN 487355 oxalate salt dinitromethane solvate occurred. Excess supernatant was removed with a syringe, and the remaining sample was dried under nitrogen.

TGA analysis of the LSN 487355 oxalate salt dinitromethane solvate showed about a 33.3 percent weight loss between room temperature and about 150 degrees C. with a heating rate of 10 degrees C./minute.

DSC analysis of the LSN 487355 oxalate salt dinitromethane solvate showed endothermic transitions at about 85.9 and 177.3 degrees C. with a heating rate of 10 degrees C./minute.

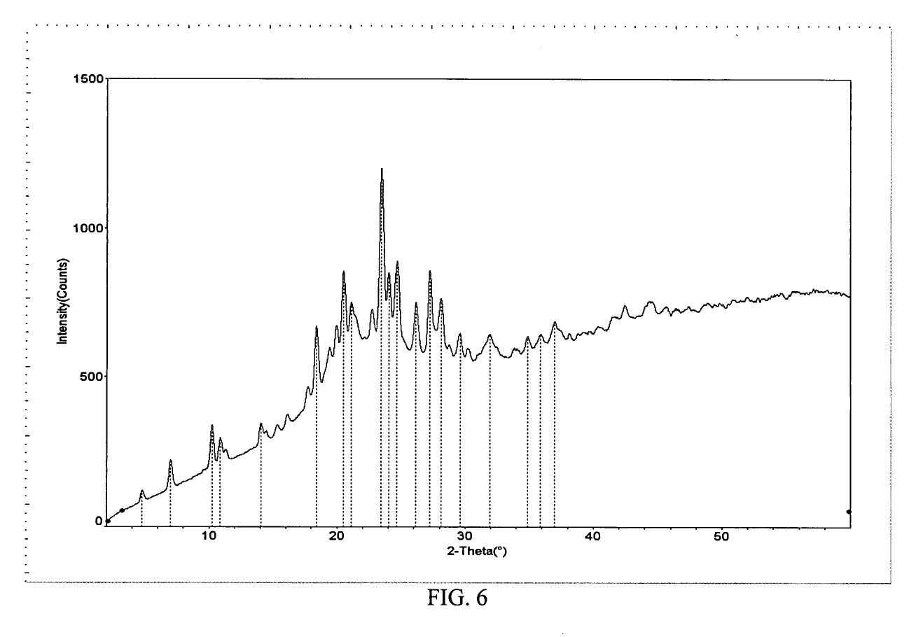

PXRD analysis of the LSN 487355 oxalate salt dinitromethane solvate was completed. The LSN 487355 oxalate salt dinitromethane solvate can be characterized by any one, any two, any three, any four, any five, or any six or more of the peaks in FIG. 6 including, but not limited to, 7.01, 10.25, 18.41, 20.51, 23.47, 24.03, 24.67, 26.17, 27.25, 28.11 and 29.61 degrees 2-theta.

### EXAMPLE 5

LSN 487355 Nitrate Salt

LSN 487355 was dissolved in nitric acid prior to combination with a mixture of toluene and isopropyl acetate. A vortex was used to mix the above materials followed by heating in a hot water bath until the contents were clear. The mixture was then cooled to 5 degrees C. for several hours during which time crystallization of the LSN 487355 nitrate salt occurred. Excess supernatant was removed with a syringe, and the remaining sample was dried under nitrogen.

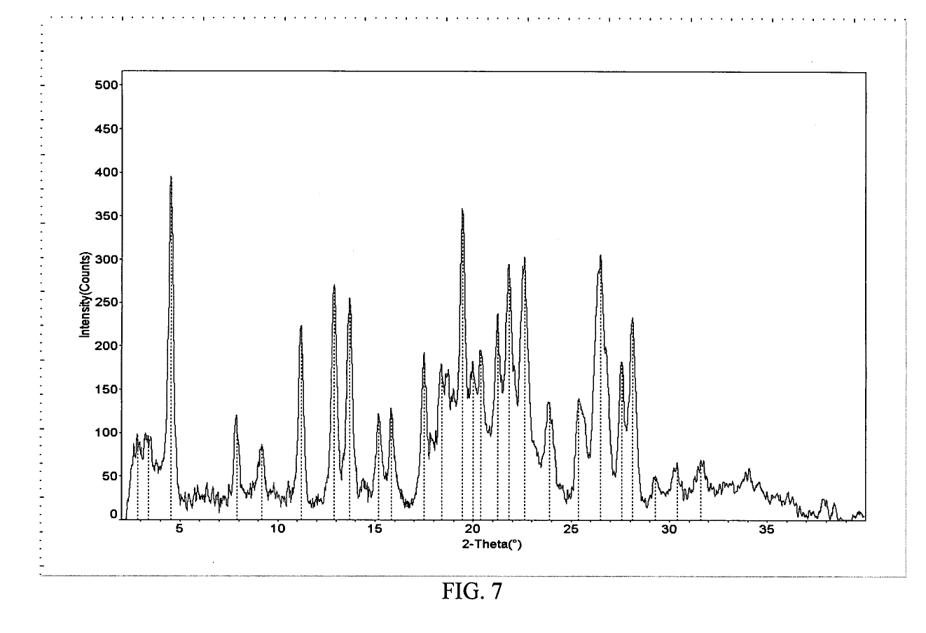

PXRD analysis of the LSN 487355 nitrate salt was completed. The LSN 487355 nitrate salt can be characterized by any one, any two, any three, any four, any five, or any six or more of the peaks in FIG. 7 including, but not limited to, 4.53, 7.91, 11.21, 12.91, 13.69, 19.45, 21.27, 21.83, 22.63, and 26.51 degrees 2-theta.

### EXAMPLE 6

LSN 487355 Form from Methanol in the Presence of Strong Acid

LSN 487355 was dissolved in methanesulfonic acid prior to combination with methanol. A vortex was used to mix the above materials followed by heating in a hot water bath until the contents were clear. The mixture was then cooled to 5 degrees C. for several hours during which time crystallization occurred. Excess supernatant was removed with a syringe, and the remaining sample was dried under nitrogen. This method was also completed with a mixture of methanol and water in place of methanol.

A similar method was also completed as above with HBr in place of methanesulfonic acid.

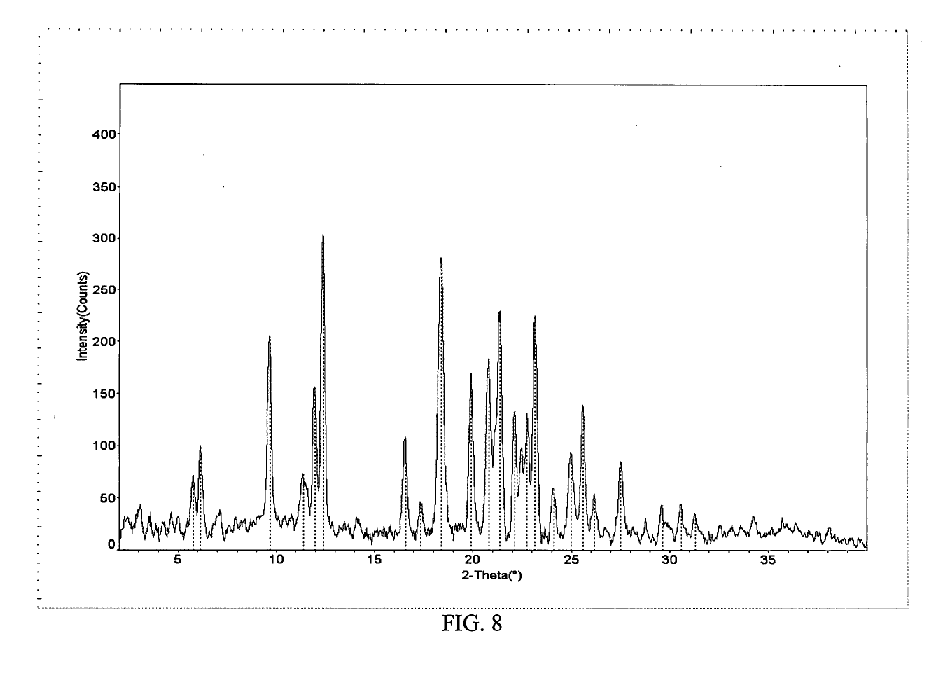

PXRD analysis of the crystallized form was completed. The form can be characterized by any one, any two, any three, any four, any five, or any six or more of the peaks in FIG. 8 including, but not limited to, 9.69, 11.95, 12.39, 18.41, 19.93, 20.81, 21.37, 22.13, 23.15, and 25.59 degrees 2-theta.

### EXAMPLE 7

LSN 487355:Fumaric Acid Co-Crystal

LSN 487355 was dissolved in nitromethane prior to combination with 1.05 equivalents of fumaric acid. A vortex was used to mix the above materials followed by heating in a hot water bath until the contents were clear. The mixture was then cooled to 5 degrees C. for several hours during which time crystallization occurred. Excess supernatant was removed with a syringe, and the remaining sample was dried under nitrogen.

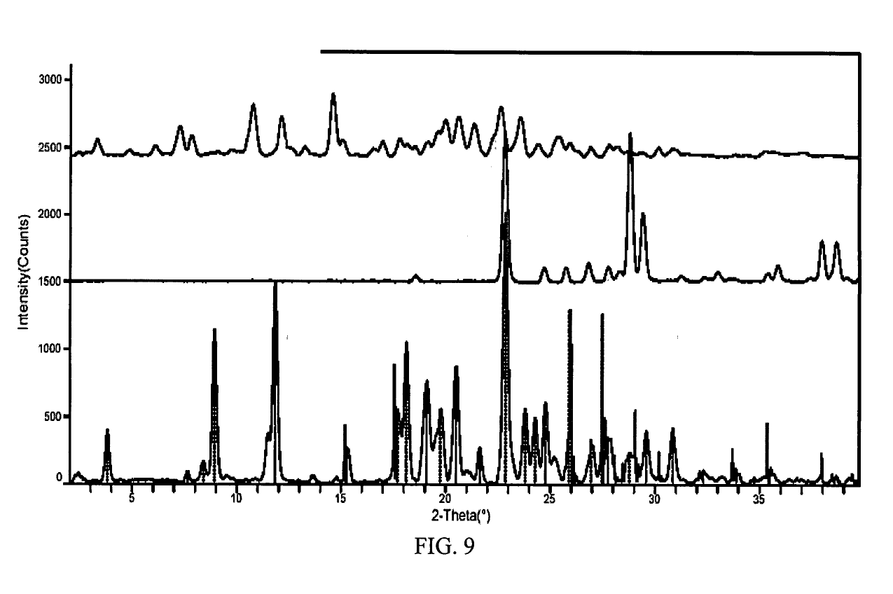

PXRD analysis of the LSN 487355:fumaric acid co-crystal was completed. The LSN 487355:fumaric acid co-crystal can be characterized by any one, any two, any three, any four, any five, or any six or more of the peaks in FIG. 9 (bottom) including, but not limited to, 3.81, 8.93, 11.83, 17.71, 18.11, 19.09, 19.74, 20.49, 22.87, 25.90, and 27.43 degrees 2-theta.

### EXAMPLE 8

LSN 487355 DMSO Solvate

3.60 mg ethanedisulfonic acid dihydrate was aded to 0.089 mL THF and mixed thoroughly. 10 microliters of this solution was combined with 10 microliters dimethyl sulfoxide (DMSO), and the resulting solution was combined with LSN 487355 powder, so that the molar ratio was 1:2 ethanedisulfonic acid:LSN 487355. The vial was crimped and equilibrated at room temperature for 2 hours, then at 5 degrees C. overnight. The resulting crystals were isolated and characterized by PXRD.

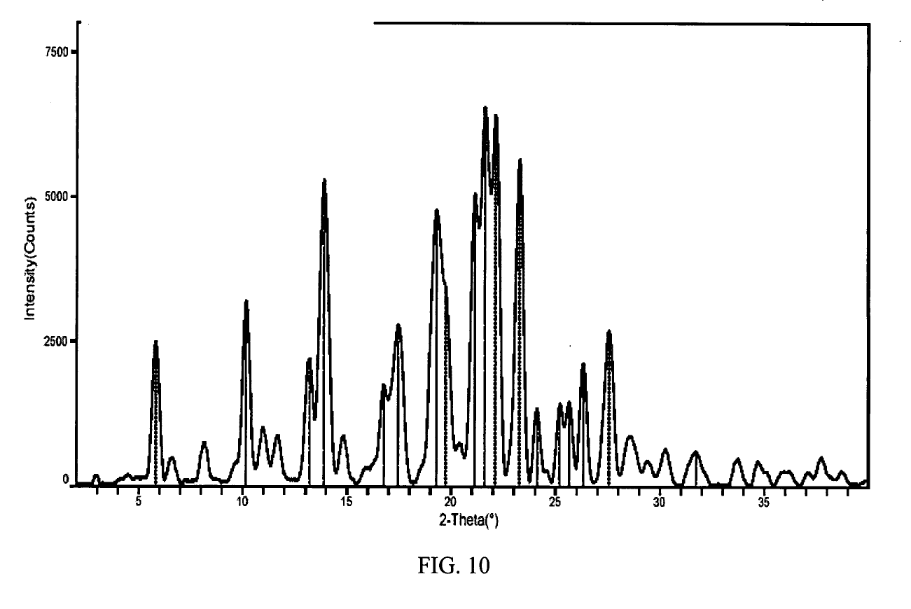

PXRD analysis of the LSN 487355 dimethyl sulfoxide (DMSO) solvate was completed. The LSN 487355 DMSO solvate can be characterized by any one, any two, any three, any four, any five, or any six or more of the peaks in FIG. 10 including, but not limited to, 5.81, 10.15, 13.21, 13.89, 16.77, 17.45, 19.29, 19.73, 21.13, 21.61, 22.11, 23.25, and 27.57 degrees 2-theta.

### EXAMPLE 9

LSN 487355 Methyl tert-butyl Ether Solvate

20 microliters of LSN 487355 stock solution (50 mg/mL in THF) was dispensed to a vial and dried. 3 microliters of ethanesulfonic acid stock solution (1 molar equivalent, 0.74 M in THF) was dispensed to the vial. 47 microliters of ethyl acetate was dispensed and the vial was crimped. After heating to 75 degrees C. and cooling to 5 degrees C., the vial was opened and solvent was evaporated with flowing nitrogen. Crystals were harvested after the vial was left overnight.

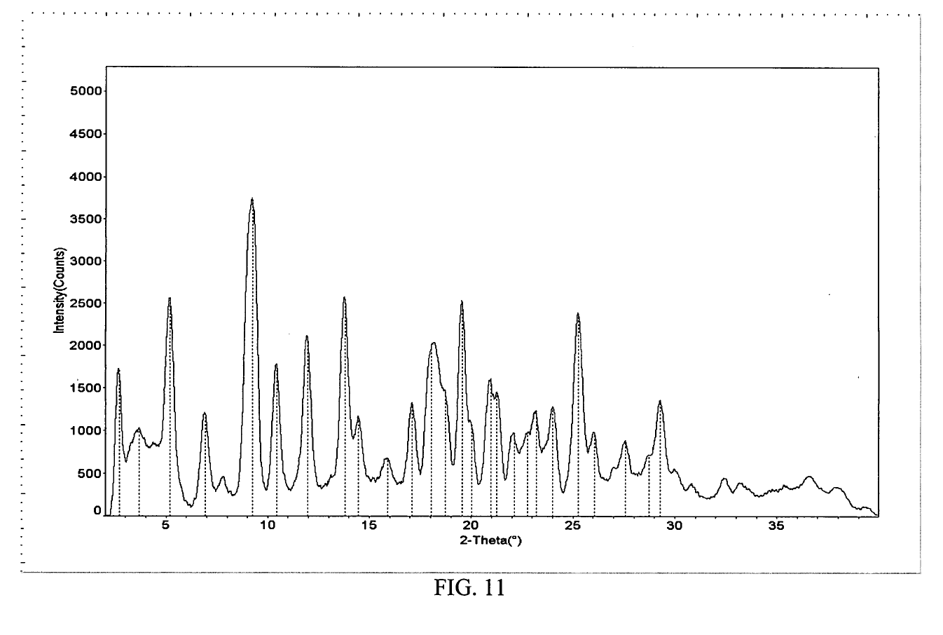

PXRD analysis of the LSN 487355 methyl tert-butyl ether solvate was completed. The LSN 487355 methyl tert-butyl ether solvate can be characterized by any one, any two, any three, any four, any five, or any six or more of the peaks in FIG. 11 including, but not limited to, 2.69, 5.19, 9.25, 10.45, 11.95, 13.79, 17.11, 18.05, 19.55, 20.97, 23.19, 24.01, 25.27, and 29.29 degrees 2-theta.

### EXAMPLE 10

LSN 487355 Formamide Solvate

Nitric acid solution in nitromethane was prepared by adding 280 mg nitric acid (68-70%) to 4.65 g nitromethane (0.73 mol/L solution). To 12.8 mg LSN 487355 was added 200 microliters of nitromethane and 47 microliters nitric acid solution. The resulting LSN 487355-nitric acid solution in nitromethane was heated in hot water until a clear solution was obtained. 20 microliters of the LSN 487355-nitric acid solution was added to 80 microliters formamide in a small tapered glass vial. The vial was crimped and equilibrated at 5 degrees C. After 3 days, the vial was uncrimped and evaporated in a turbovap under flowing nitrogen. Crystals were observed, and characterized by PXRD.

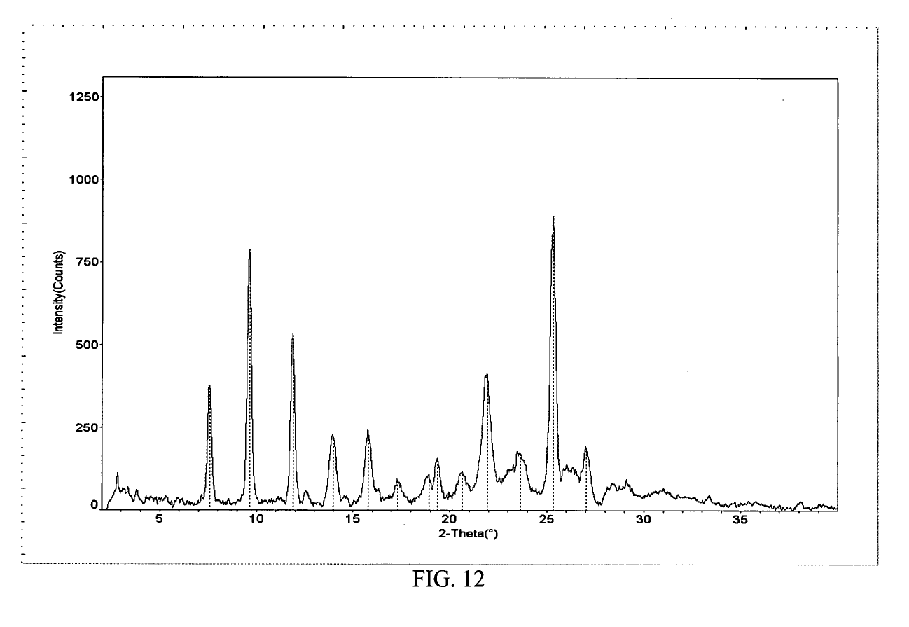

PXRD analysis of the LSN 487355 formamide solvate was completed. The LSN 487355 formamide solvate can be characterized by any one, any two, any three, any four, any five, or any six or more of the peaks in FIG. 12 including, but not limited to, 7.59, 9.65, 11.87, 13.97, 15.79, 21.93, 25.35, and 27.05 degrees 2-theta.

### EXAMPLE 11

Formulation Studies of LSN 487355

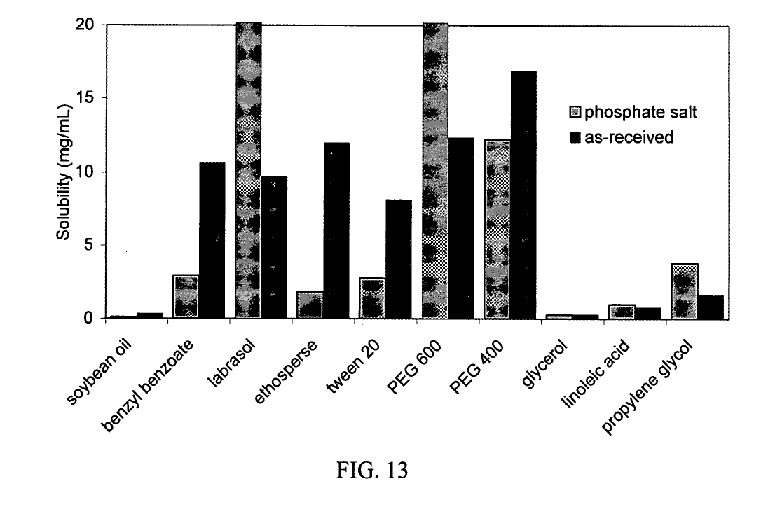

A single excipient screen was completed in an attempt to find an appropriate excipient for a liquid formulation of LSN 487355. The following excipients were included in the screen: benzyl alcohol, isopropanolamine, vitamin E TPGS, PEG-8 caprylic/capric glyceride (Labrasol), diethylene glycol monoethyl ether (Transcutol P), benzyl benzoate, PEG-600, PEG-20, polyoxyethylene 20 glycerol oleate, PEG 60 almond glycerides, PEG-300, polyoxyl 40 stearate, PEG-200, polyoxyl 35 castor oil, polysorbate 20, polyoxyethylene 26 glycerin, ethylene glycol monoethyl ether, propylene glycol monocaprylate, polyoxyl 40 castor oil, PEG-32 glyceryl laurate, polyoxyl 20 stearate, polysorbate 60, polysorbate 40, polysorbate 80, PEG-400, polyoxyl 40 hydrogenated castor oil, acetylated monoglycerides, monoolein:propylene glycol (90:10), mono-/diglyceride from coconut oil (C8/C10), caprylic/capric triglyceride, C8/C10 diesters of propylene glycol of coconut oil, castor oil, coconut oil, corn oil, cottonseed oil, soybean oil, diacetylated monoglycerides, ethylene glycol, gelucire 33/01, glycerin, glyceryl oleate, glyceryl linoleate, glyceryl ricinoleate, hydrogenated coconut oil, oleoyl macrogol-6 glycerides (apricot kernel oil PEG-6 ester), linoleoyl macrogol-6 glycerides (corn oil PEG-6 esters), propylene glycol monolaurate, lecithin (high HLB), lecithin (low HLB), linoleic acid, mineral oil, and myristyl alcohol. Binary and tertiary excipient screens were also completed with excipient ratios, for example, of 75:25, 50:50, 34:33:33, and 66:17:17. FIG. 13 compares the solubility of LSN 487355 free base (as received) and the solubility of the phosphate salt of LSN 487355 in various excipients. The phosphate salt has a solubility of at least 20 mg/mL in two excipients, labrasol and PEG-600.

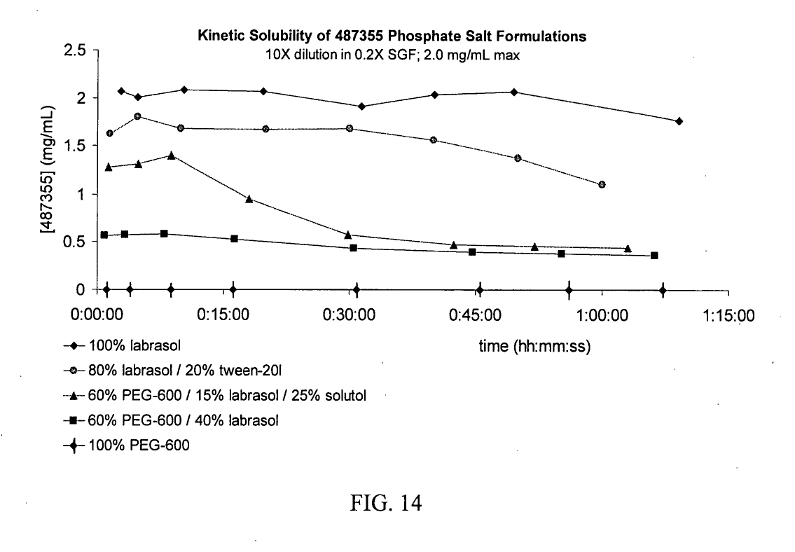

A dissolution scheme was used to measure the kinetic dissolution of various formulations of LSN 487355. For example, a first sample was prepared with a 30 mg/mL concentration of LSN 487355 phosphate salt and a 20 mg/mL concentration of LSN 487355 free base in 1 mL PEG-600, another sample was prepared with a 30 mg/mL concentration of LSN 487355 phosphate salt and a 20 mg/mL concentration of LSN 487355 free base in 1 mL 60:40 (v/v) PEG-60:labrasol, and a third sample was prepared with a 30 mg/mL concentration of LSN 487355 phosphate salt and a 20 mg/mL concentration of LSN 487355 free base in 1 mL 60:25:15 (v/v) PEG-600:labrasol:solutol. The samples were diluted by a factor of 10 with 0.2× simulated gastric fluid (SGF). The kinetic dissolution of these samples were monitored over a 1 hour period and the results can be found in FIG. 14. The formulation in labrasol maintained an LSN 487355 concentration of about 2 mg/mL over the entire duration of the experiment. A formulation containing 80:20 (v/v) labrasol:tween 20 (polysorbate 20) maintained a concentration of LSN 487355 above 1.5 mg/mL for about 30 minutes before the concentration began to decrease.

### EXAMPLE 12

LSN 509207 Form II Polymorph

2.0 mg of dissolved LSN 509207 was added using THF as a carrier solvent. The solvent was then evaporated under nitrogen. 2.2 molar equivalents of dissolved succinic acid was added using methanol as a carrier solvent. The solvent was then evaporated under nitrogen. Processing solvent was 100 microliters of nitromethane and 50 microliters of isopropanol. Sample was heated to 75C for 2 hours followed by incubation at 5 degrees C. for up to 1 week. Sample was dried and followed up with powder x-ray diffraction.

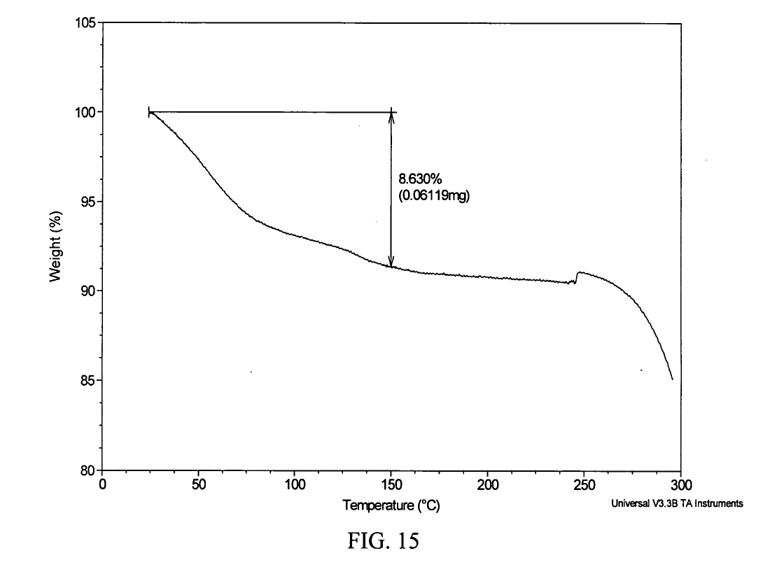

TGA analysis of the LSN 509207 Form II Polymorph is shown in FIG. 15, and showed about an 8.6 percent weight loss between room temperature and about 150 degrees C. with a heating rate of 10 degrees C./minute.

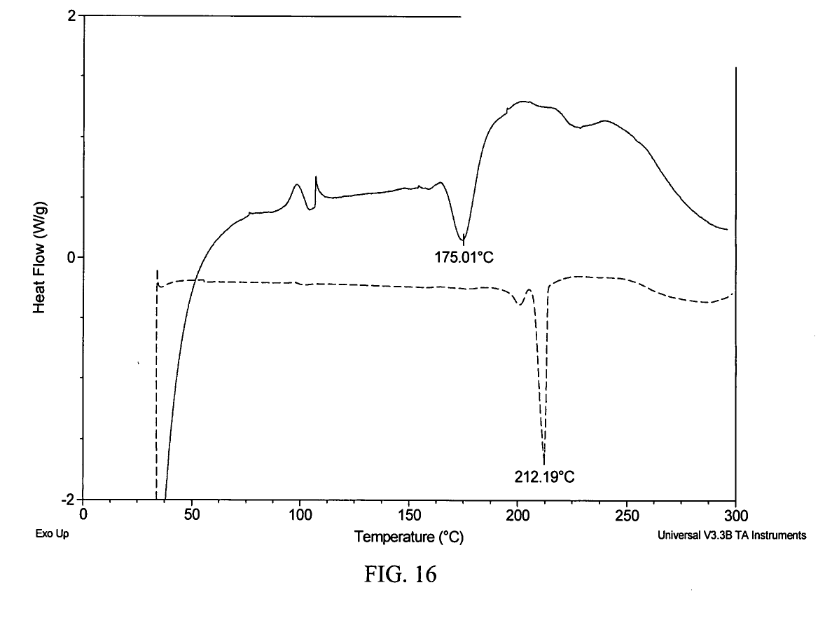

DSC analysis of the LSN 509207 Form II Polymorph is shown in FIG. 16 (solid line), and showed an endothermic transition at about 175 degrees C. with a heating rate of 10 degrees C./minute.

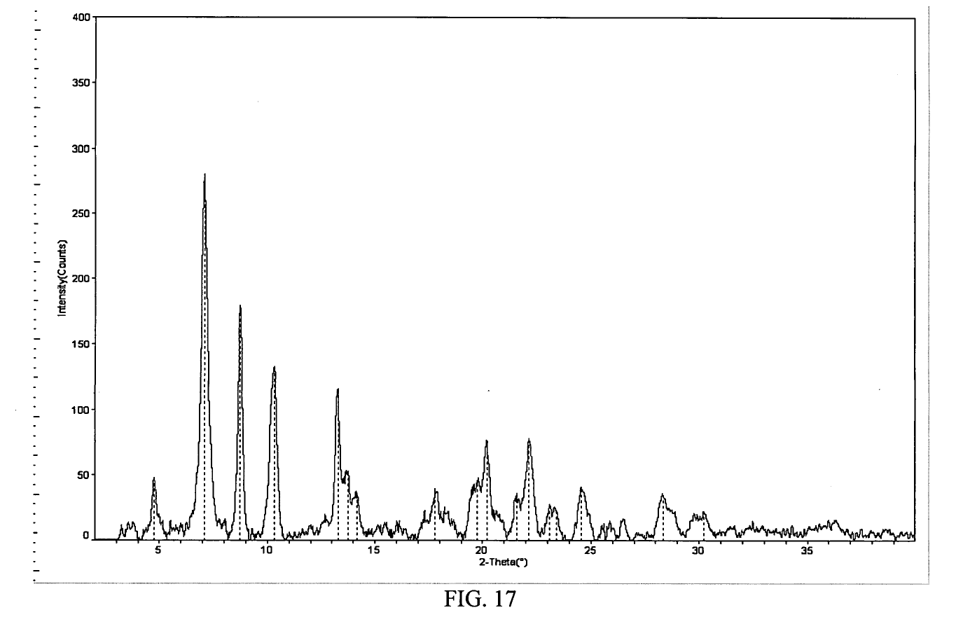

PXRD analysis of the LSN 509207 Form II Polymorph was completed. The LSN 509207 Form II Polymorph can be characterized by any one, any two, any three, any four, any five, or any six or more of the peaks in FIG. 17 including, but not limited to 4.77, 7.07, 8.75, 10.33, 13.27, 13.75, 14.19, 17.79, 19.77, 20.19, 21.57, 22.15, 23.11, 24.55, 28.35, and 30.23 degrees 2-theta.

### EXAMPLE 13

LSN 509207 Form III Polymorph

2.0 mg of dissolved LSN 509207 was added using THF as a carrier solvent. The solvent was then evaporated under nitrogen. 2.2 molar equivalents of dissolved tris base was added using water as a carrier solvent. The solvent was then evaporated under nitrogen. Processing solvent was 150 microliters of 1,2-dichloroethane. Sample was heated to 75 degrees C. for 2 hours followed by incubation at 5 degrees C. for up to 1 week. Sample was dried and followed up with powder x-ray diffraction.

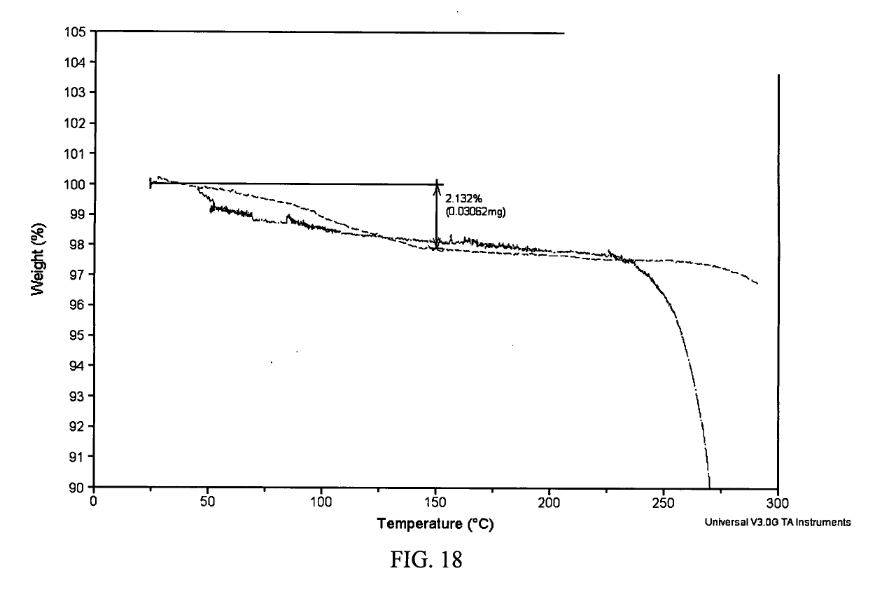

TGA analysis of the LSN 509207 Form III Polymorph is shown in FIG. 18 (dashed line), and showed about a 2.1 percent weight loss between room temperature and about 150 degrees C. with a heating rate of 10 degrees C./minute.

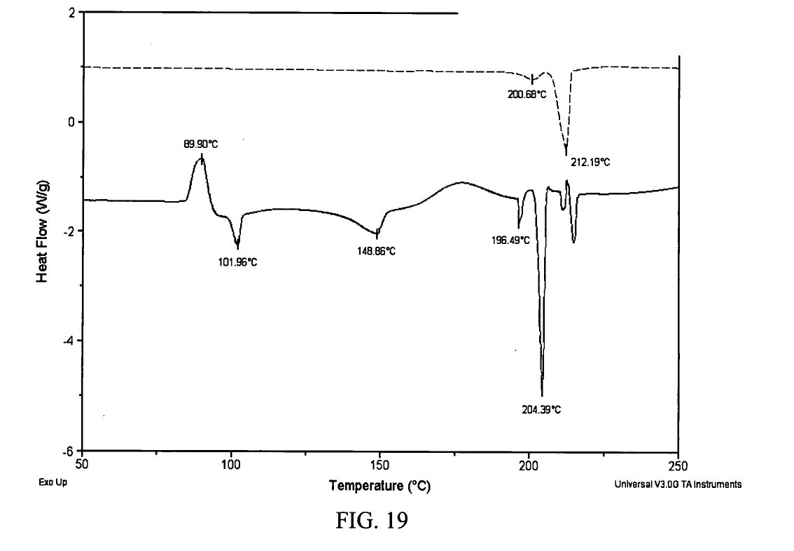

DSC analysis of the LSN 509207 Form III Polymorph is shown in FIG. 19 (solid line), and showed endothermic transitions at about 102,149,196 and 204 degrees C. with a heating rate of 10 degrees C./minute.

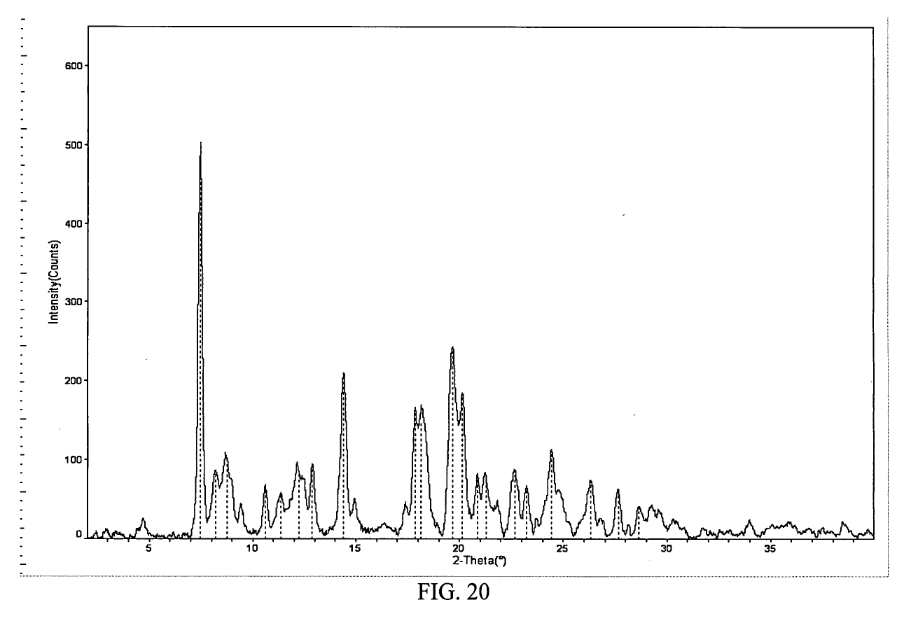

PXRD analysis of the LSN 509207 Form III Polymorph was completed. The LSN 509207 Form III Polymorph crystallized in 1,2-dichloromethane in presence of tris base can be characterized by any one, any two, any three, any four, any five, or any six or more of the peaks in FIG. 20 including, but not limited to, 7.47, 8.19, 8.75, 10.59, 11.35, 12.25, 12.89, 14.41, 17.85, 18.15, 19.69, 20.15, 20.85, 21.27, 22.65, 23.25, 24.43, 26.31, and 27.65 degrees 2-theta.

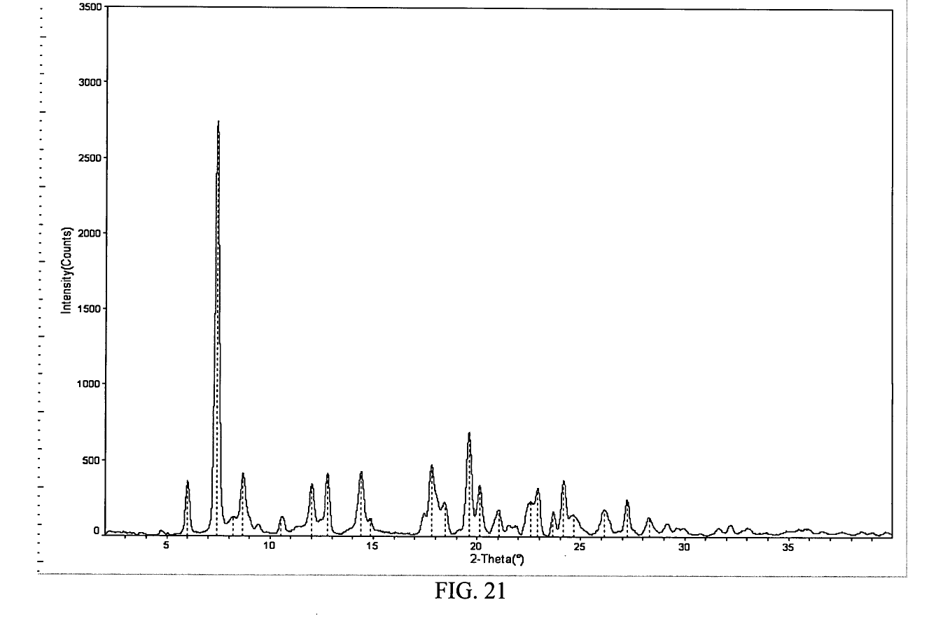

The LSN 509207 Form III Polymorph crystallized in acetone can be characterized by any one, any two, any three, any four, any five, or any six or more of the peaks in FIG. 21 including, but not limited to 6.01, 7.41, 8.69, 12.01, 12.79, 14.41, 17.81, 19.61, 20.13, 22.91, 24.17, and 27.25 degrees 2-theta.

### EXAMPLE 14

LSN 509207 Form IV Polymorph

2.0 mg of dissolved LSN 509207 was added using THF as a carrier solvent. The solvent was then evaporated under nitrogen. 0.55 molar equivalents of dissolved nicotinamide was added using methanol as a carrier solvent. The solvent was then evaporated under nitrogen. Processing solvent was 100 microliters of acetonitrile and 50 microliters of isopropanol. Sample was heated to 75 degrees C. for 2 hours followed by incubation at 5 degrees C. for up to 1 week. Sample was dried and followed up with powder x-ray diffraction.

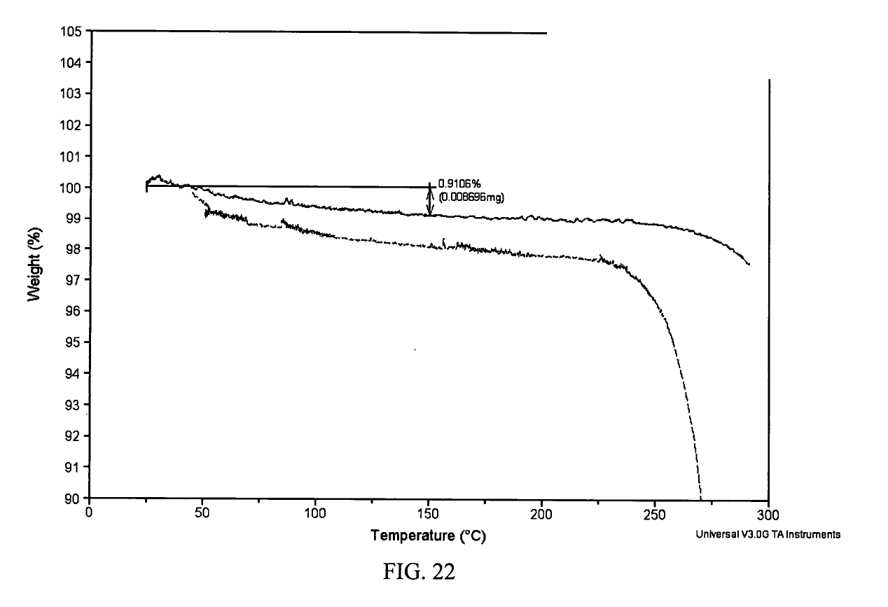

TGA analysis of the LSN 509207 Form IV Polymorph is shown in FIG. 22 (solid line), and showed about a 0.9 percent weight loss between room temperature and about 150 degrees C. with a heating rate of 10 degrees C./minute.

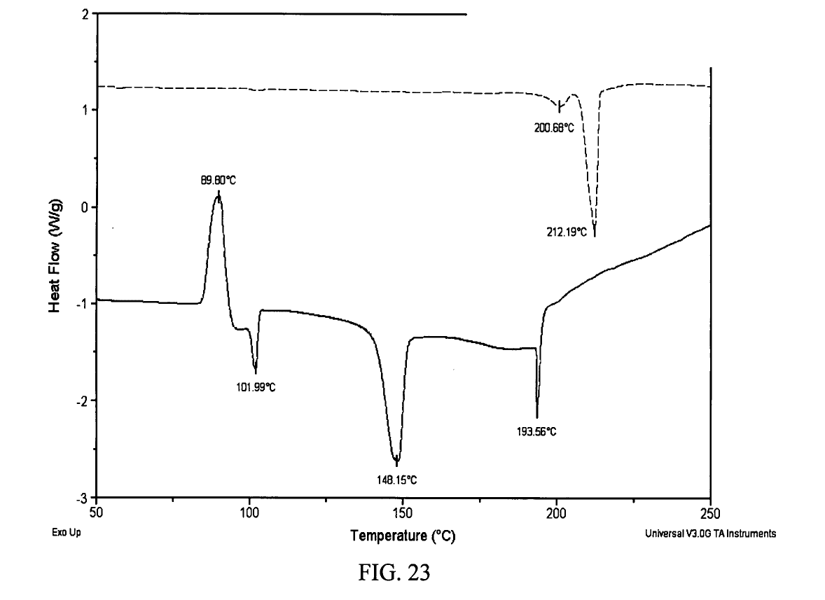

DSC analysis of the LSN 509207 Form IV Polymorph is shown in FIG. 23 (solid line), and showed endothermic transitions at about 102,148 and 194 degrees C. with a heating rate of 10 degrees C./minute.

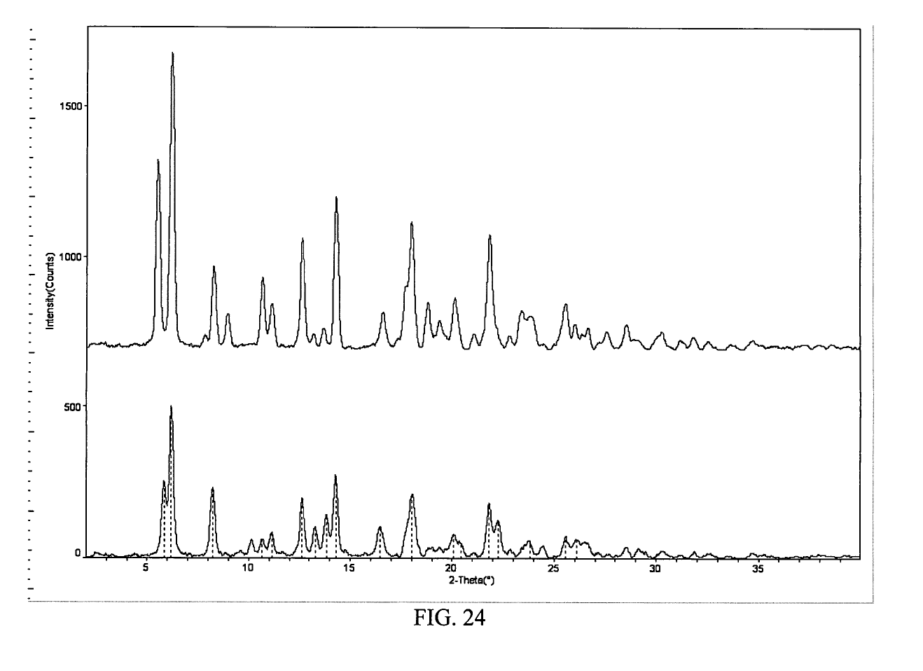

PXRD analysis of the LSN 509207 Form IV Polymorph was completed. The LSN 509207 Form IV Polymorph crystallized in nitromethane/isopropanol solution in presence of nicotinamide can be characterized by any one, any two, any three, any four, any five, or any six or more of the peaks in FIG. 24 (bottom pattern) including, but not limited to 5.83, 6.19, 8.23, 12.63, 14.29, 18.03, 21.83, 23.81, and 25.57 degrees 2-theta.

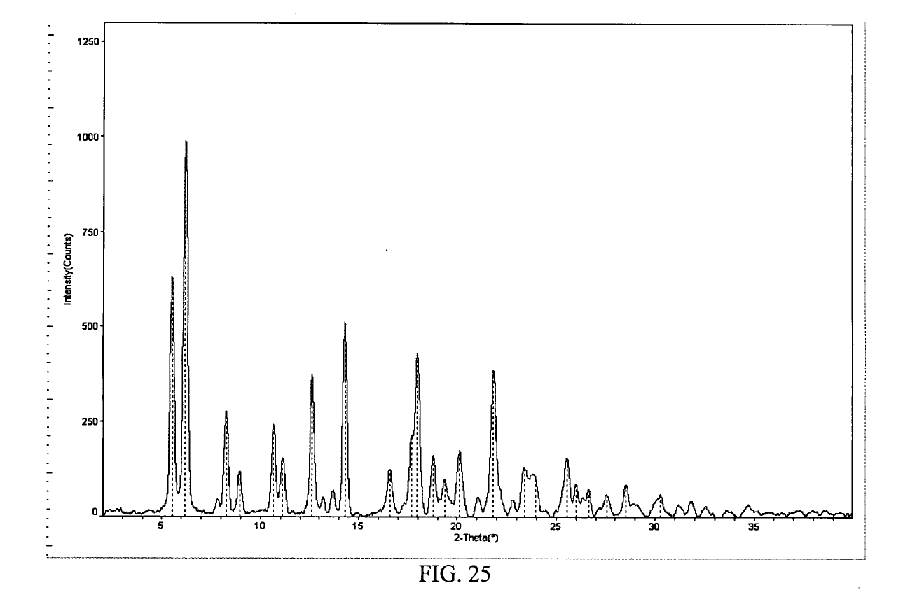

PXRD analysis of the LSN 509207 Form IV Polymorph was completed. The LSN 509207 Form IV Polymorph crystallized in acetonitrile can be characterized by any one, any two, any three, any four, any five, or any six or more of the peaks in FIG. 25 including, but not limited to 5.51, 6.19, 8.27, 8.97, 10.67, 11.13, 12.63, 14.29, 16.61, 17.97, 18.79, 20.13, 23.39, 25.57, and 28.55 degrees 2-theta.

### EXAMPLE 15

LSN 509207 Form V Polymorph

2.0 mg of dissolved LSN 509207 was added using THF as a carrier solvent. The solvent was then evaporated under nitrogen. 0.55 molar equivalents of dissolved acetaminophen was added using methanol as a carrier solvent. The solvent was then evaporated under nitrogen. Processing solvent was 100 microliters of heptane and 50 microliters of 1,2-dichloroethane. Sample was heated to 75 degrees C. for 2 hours followed by incubation at 5 degrees C. for up to 1 week. Sample was dried and followed up with powder x-ray diffraction.

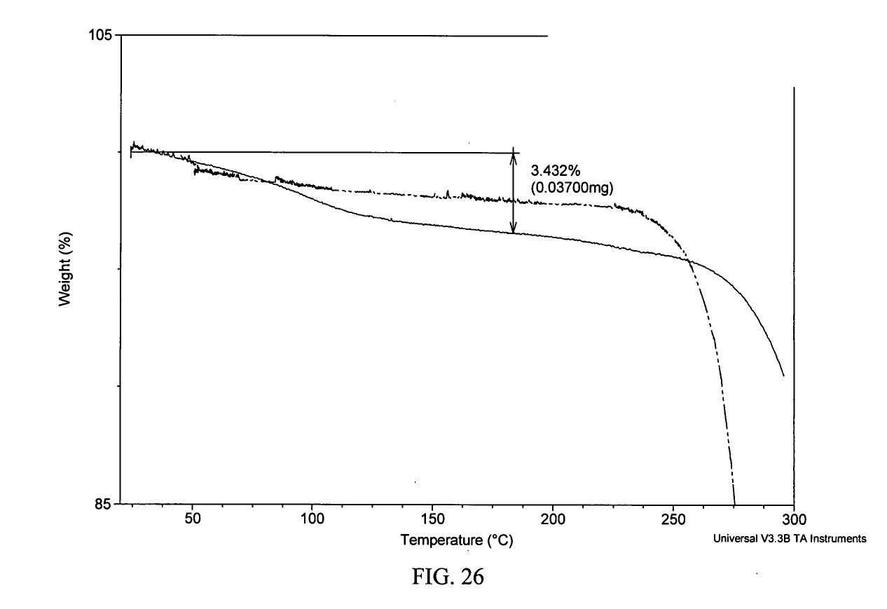

TGA analysis of the LSN 509207 Form V Polymorph is shown in FIG. 26 (solid line), and showed about a 3.4 percent weight loss between room temperature and about 150 degrees C. with a heating rate of 10 degrees C./minute.

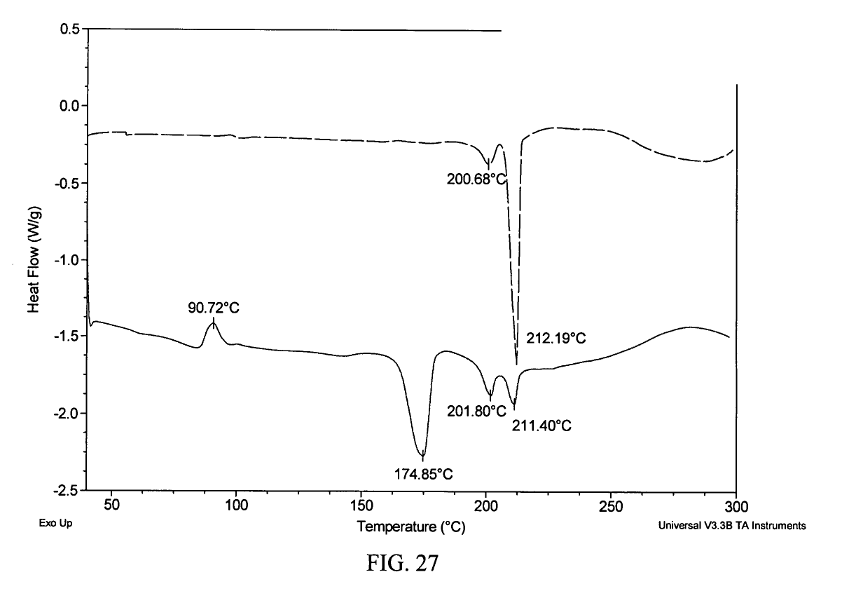

DSC analysis of the LSN 509207 Form V Polymorph is shown in FIG. 27 (solid line), and showed endothermic transitions at about 175, 202, and 211 degrees C. with a heating rate of 10 degrees C./minute.

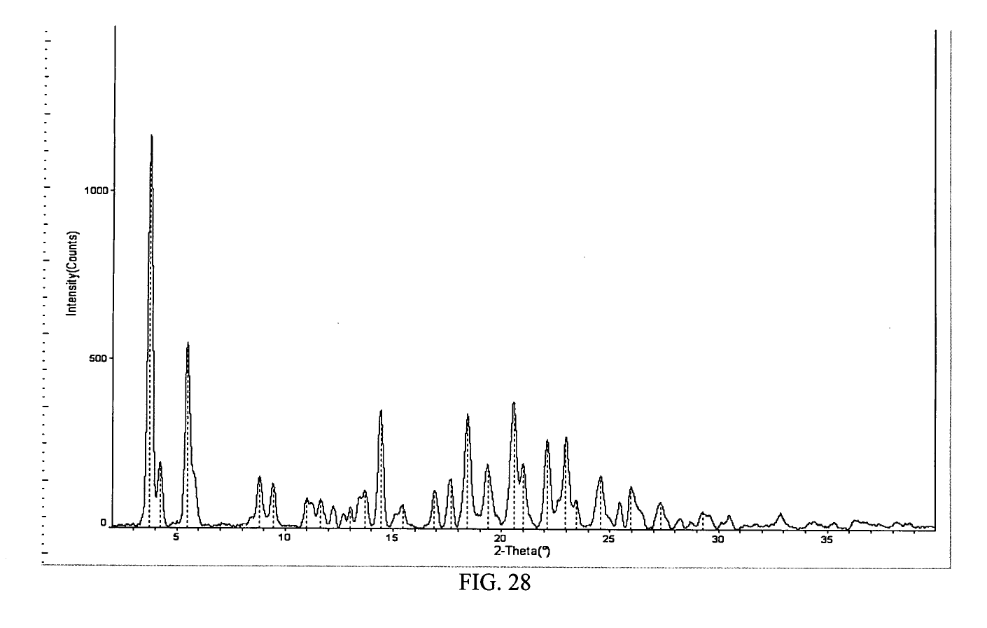

PXRD analysis of the LSN 509207 Form V Polymorph was completed. The LSN 509207 Form V Polymorph can be characterized by any one, any two, any three, any four, any five, or any six or more of the peaks in FIG. 28 including, but not limited to, 3.73, 4.21, 5.49, 8.81, 9.43, 10.99, 11.63, 13.71, 14.43, 16.91, 17.67, 18.43, 19.37, 20.57, 21.01, 22.11, 22.97, 24.59, and 25.97 degrees 2-theta.

### EXAMPLE 16

LSN 509207:1-hydroxy-2-naphthoic Acid Co-Crystal

2.0 mg of dissolved LSN 509207 was added using THF as a carrier solvent. The solvent was then evaporated under nitrogen. 0.55 molar equivalents of dissolved 1-hydroxy-2-naphthoic acid was added using tetrahydofuran as a carrier solvent. The solvent was then evaporated under nitrogen. Processing solvent was 150 microliters of 1,2-dichloroethane. Sample was heated to 75 degrees C. for 2 hours followed by incubation at 5 degrees C. for up to 1 week. Sample was dried and followed up with powder x-ray diffraction.

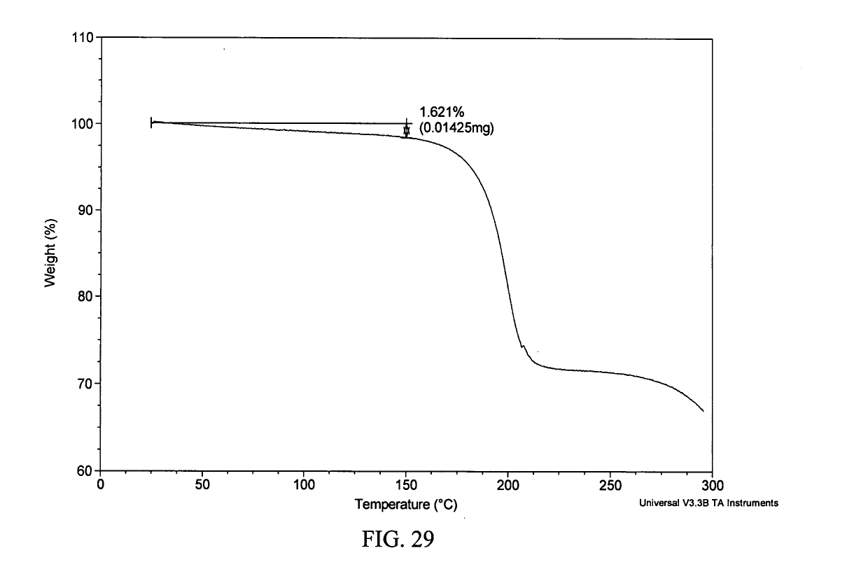

TGA analysis of the LSN 5092071-hydroxy-2-naphthoic acid co-crystal is shown in FIG. 29, and showed about a 1.6 percent weight loss between room temperature and about 150 degrees C. with a heating rate of 10 degrees C./minute.

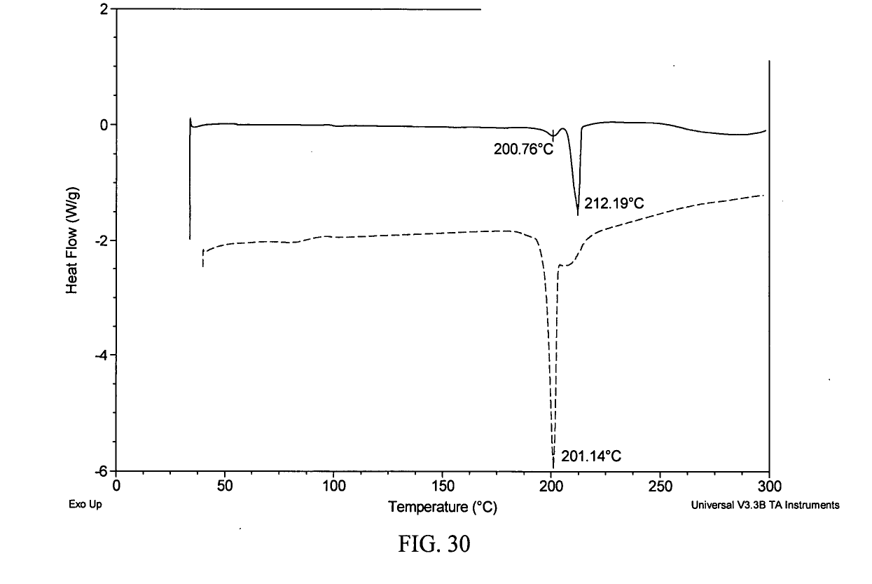

DSC analysis of the LSN 5092071-hydroxy-2-naphthoic acid co-crystal is shown in FIG. 30 (dashed line), and showed an endothermic transition at about 201 degrees C. with a heating rate of 10 degrees C./minute.

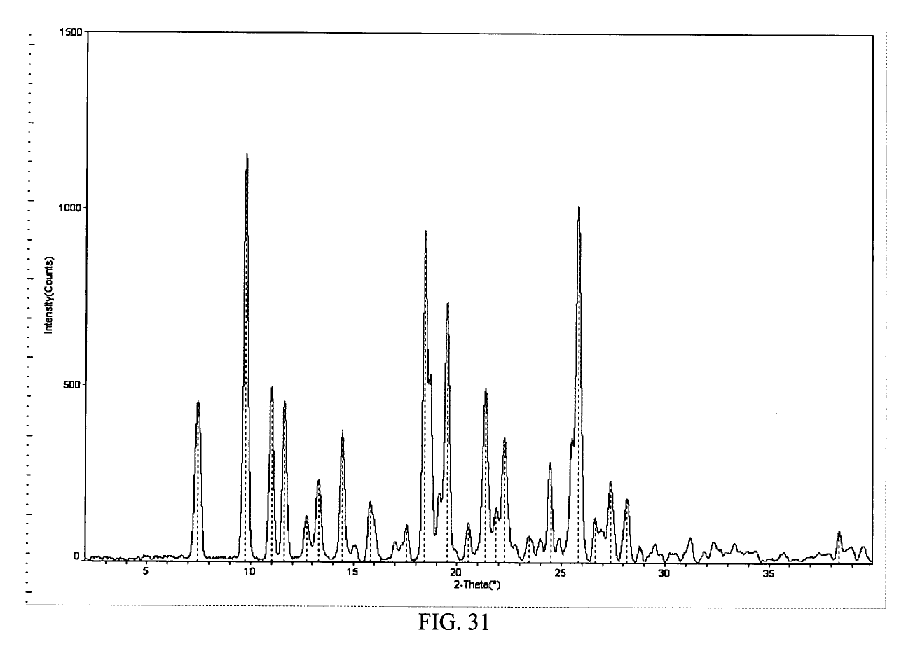

PXRD analysis of the LSN 509207:1-hydroxy-2-naphthoic acid co-crystal was completed. The LSN 509207:1-hydroxy-2-naphthoic acid co-crystal can be characterized by any one, any two, any three, any four, any five, or any six or more of the peaks in FIG. 31 including, but not limited to 7.47,9.75, 11.01, 11.63, 12.71, 13.31, 14.45 15.81, 18.43, 19.51, 21.35, 22.27, 24.49, 25.81, 27.39, and 28.17 degrees 2-theta.

### EXAMPLE 17

LSN 509207 Tetrahydrofuran Solvate

0.87 mg of dry LSN 509207 was added to a vial. 10 microliters of tetrohydrofuran was added. Sample was heated to 50 degrees C. for 2 hours followed by incubation at 5 degrees C. overnight. Sample was dried and followed up with powder x-ray diffraction.

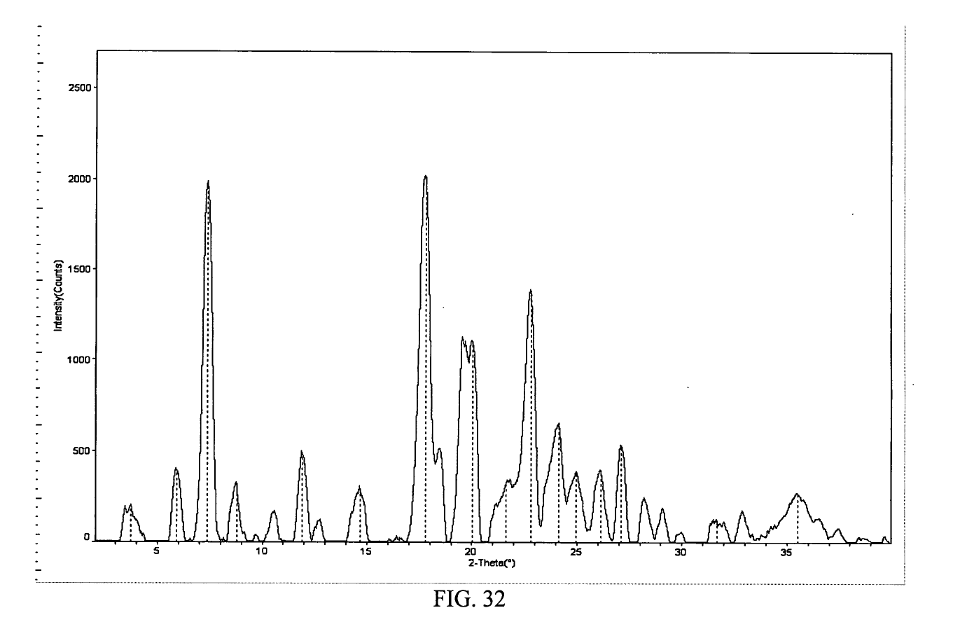

PXRD analysis of the LSN 509207 tetrahydrofuran solvate was completed. The LSN 509207 tetrahydrofuran solvate can be characterized by any one, any two, any three, any four, any five, or any six or more of the peaks in FIG. 32 including, but not limited to 3.73, 5.90, 7.38, 8.77, 11.88, 14.67, 17.77, 20.02, 21.65, 22.81, 24.12, 24.97, 26.14, 27.09, and 35.50 degrees 2-theta.

### EXAMPLE 18

LSN 509207 Acetic Acid Solvate

0.86 mg of dry LSN 509207 was added to a vial. 10 microliters of acetic acid was added. Sample was heated to 50 degrees C. for 2 hours followed by incubation at 5 degrees C. overnight. Sample was dried and followed up with powder x-ray diffraction.

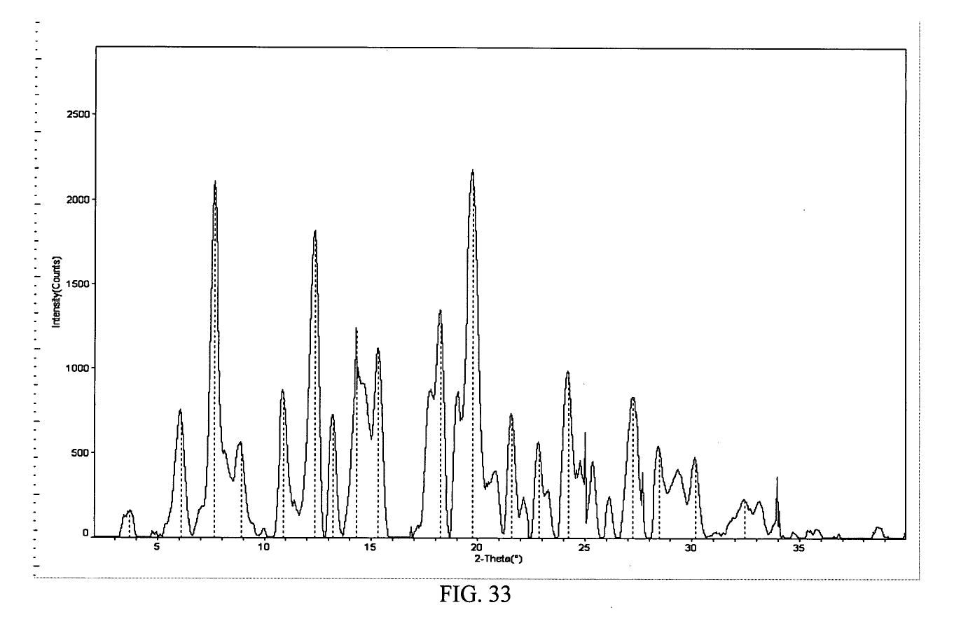

PXRD analysis of the LSN 509207 acetic acid solvate was completed. The LSN 509207 acetic acid solvate can be characterized by any one, any two, any three, any four, any five, or any six or more of the peaks in FIG. 33 including, but not limited to 3.69, 6.07, 7.65, 8.91, 10.89, 12.37, 13.22, 14.31, 15.34, 18.22, 19.75, 21.59, 22.86, 24.21, 27.23, 28.48, 30.15, and 32.44 degrees 2-theta.

### EXAMPLE 19

LSN 509207 Pyridine Solvate

1.03 mg of dry LSN 509207 was added to a vial. 10 microliters of pyridine was added. Sample was heated to 50 degrees C. for 2 hours followed by incubation at 5 degrees C. overnight. Sample was dried and followed up with powder x-ray diffraction.

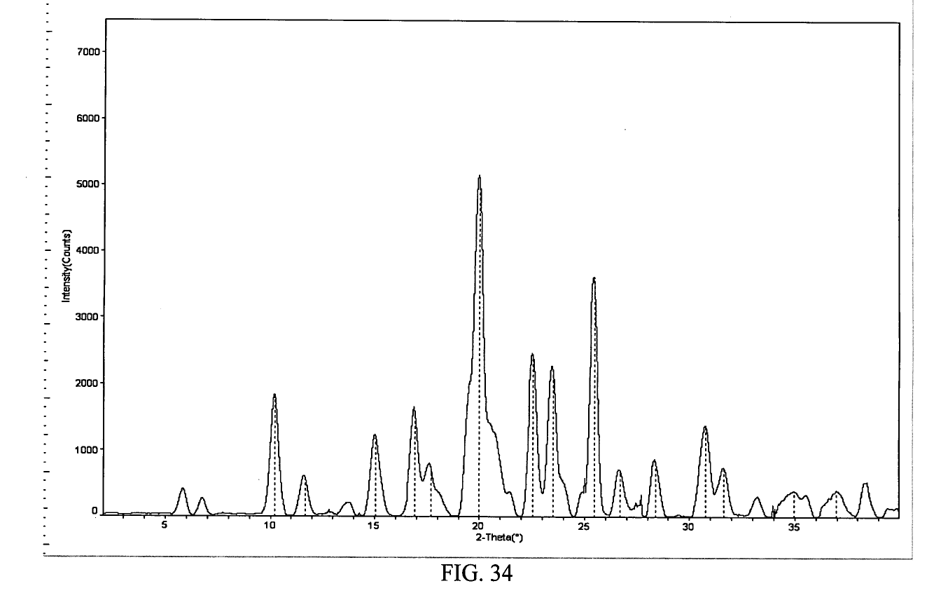

PXRD analysis of the LSN 509207 pyridine solvate was completed. The LSN 509207 pyridine solvate can be characterized by any one, any two, any three, any four, any five, or any six or more of the peaks in FIG. 34 including, but not limited to 10.21, 11.65, 15.03, 16.92, 17.68, 19.98, 22.54, 23.49, 25.47, 26.68, 28.39, 30.78, 31.63, 34.96, and 36.99 degrees 2-theta.

### EXAMPLE 20

LSN 509207 Dioxane Solvate

0.88 mg of dry LSN 509207 was added to a vial. 10 microliters of dioxane was added. Sample was heated to 50 degrees C. for 2 hours followed by incubation at 5 degrees C. overnight. Sample was dried and followed up with powder x-ray diffraction.

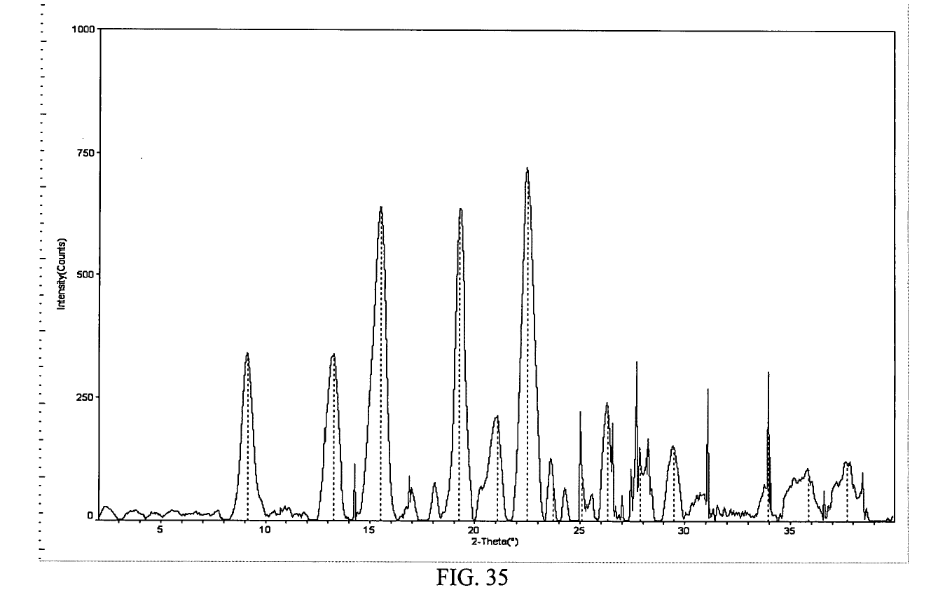

PXRD analysis of the LSN 509207 dioxane solvate was completed. The LSN 509207 dioxane solvate can be characterized by any one, any two, any three, any four, any five, or any six or more of the peaks in FIG. 35 including, but not limited to 9.13, 13.27, 15.52, 19.26, 21.10, 22.50, 23.73, 25.11, 26.32, 27.87, 29.47, 33.96, 35.86, and 37.71 degrees 2-theta.

### EXAMPLE 21

LSN 509207 Nitromethane Solvate

0.84 mg of dry LSN 509207 was added to a vial. 10 microliters of nitromethane was added. Sample was heated to 50 degrees C. for 2 hours followed by incubation at 5 degrees C. overnight. Sample was dried and followed up with powder x-ray diffraction.

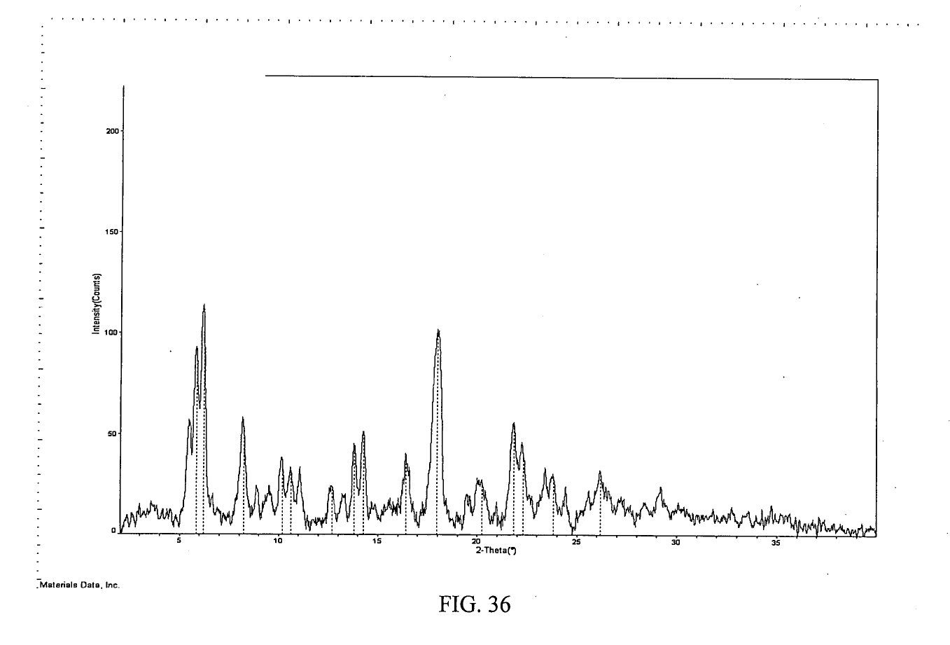

PXRD analysis of the LSN 509207 nitromethane solvate was completed. The LSN 509207 nitromethane solvate can be characterized by any one, any two, any three, any four, any five, or any six or more of the peaks in FIG. 36 including, but not limited to 5.85, 6.21, 8.23, 10.19, 10.61, 12.69, 13.81, 14.27, 16.41, 17.97, 20.25, 21.85, 22.31, 23.83, and 26.17 degrees 2-theta.

### EXAMPLE 22

LSN 509207 Methylene Chloride Solvate

0.85 mg of dry LSN 509207 was added to a vial. 20 microliters of methylene chloride was added. Sample was heated to 50 degrees C. for 2 hours followed by incubation at 5 degrees C. overnight. Sample was dried and followed up with powder x-ray diffraction.

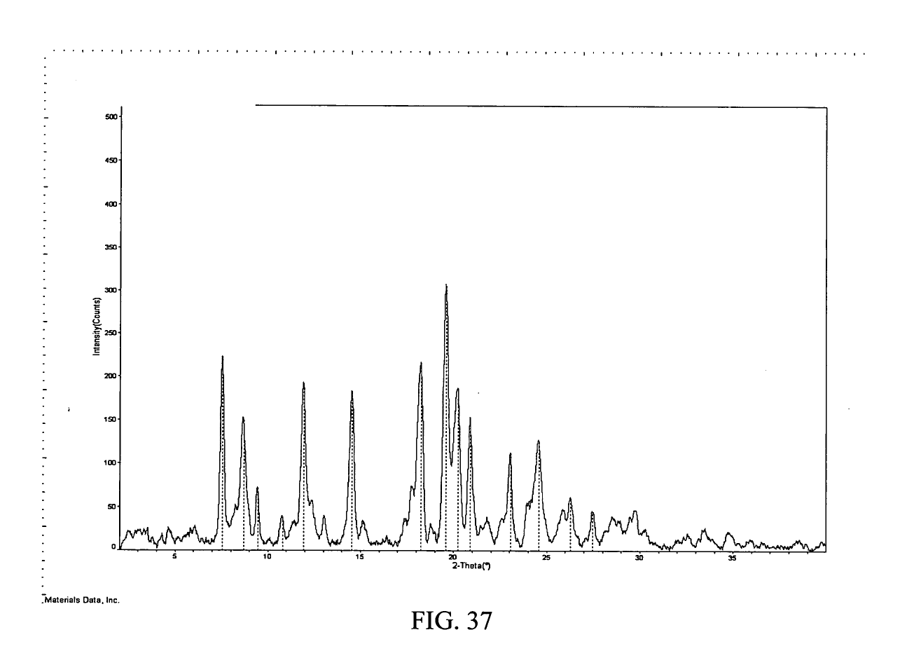

PXRD analysis of the LSN 509207 methylene chloride solvate was completed. The LSN 509207 methylene chloride solvate can be characterized by any one, any two, any three, any four, any five, or any six or more of the peaks in FIG. 37 including, but not limited to 7.55, 8.69, 9.45, 11.93, 14.53, 18.25, 19.59, 20.23, 20.89, 23.05, 24.57, 26.29, and 27.49 degrees 2-theta.

What is claimed is:

1. A form of a compound of the formula (I): 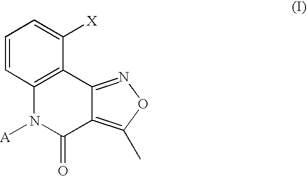
wherein:
A is substituted or unsubstituted and is 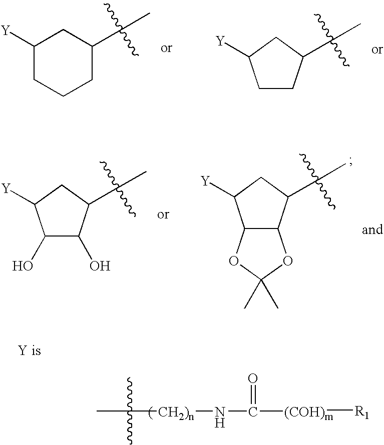
where n and m can be the same or different and are individually 0 or 1 and R₁ is substituted or unsubstituted benzene or pyridine, X is a halogen, and wherein the form (a) is formed by the reaction of a compound of formula (I) and an organic or inorganic acid in a crystallization solvent, and (b) has a lipid solubility of at least about 10 mg/mL.

2. The form of claim 1, wherein the form is:
(a) made by reacting LSN 487355 with an organic or inorganic acid in a crystallization solvent, wherein the form has a lipid solubility of approximately 10 mg/mL to approximately 150 mg/mL; or
(b) a phosphate salt made by reacting LSN 487355 with phosphoric acid.

3. The form of claim 1, formed by reacting either LSN 487355 or LSN 509207 and an organic or inorganic acid in a heated crystallization solvent to form a reaction product, and thereafter cooling the reaction product to a temperature of between about 0° C. to about 10° C. to form the form, wherein the form has a lipid solubility of between about 10 mg/ml to about 150 mg/ml.

4. The form of claim 3, wherein the crystallization solvent prior to form formation further comprises a seed crystal comprising a salt formed by the reaction of either LSN 487355 or LSN 509207 and an organic or inorganic acid.

5. A pharmaceutical formulation comprising a form of LSN 487355 or LSN 509207 and an excipient system comprising Labrasol, wherein the excipient system comprises approximately 25% to 75% by weight of the formulation, the lipid solubility of LSN 487355 or LSN 509207 in the excipient system is approximately 10 mg/ml to approximately 150 mg/ml, and wherein the formulation can be administered orally or parenterally.

6. The form of claim 3, wherein:
(a) the form is a dimethyl sulfoxide, methyl tert-butyl ether, or formamide solvate of LSN 487355;
(b) the form is a nitromethane solvate of an oxalate salt of LSN 487355 formed by the recrystallization of the oxalate salt of LSN 487355 in a crystallization solvent comprising nitromethane;
(c) the form is a dinitromethane solvate of an oxalate salt of LSN 487355 formed by the recrystallization of the oxalate salt of LSN 487355 in a crystallization solvent comprising dinitromethane;
(d) the form is a nitrate salt of LSN 487355 formed by the recrystallization of LSN 487355 in a crystallization solvent comprising toluene and isopropyl acetate;
(e) the form is a LSN 487355:fumaric acid co-crystal formed by the crystallization of LSN 487355 free base in a crystallization solvent comprising fumaric acid and nitromethane; or
(f) the form is a LSN 487355 solvate formed by the crystallization of LSN 487355 in a crystallization solvent selected from the group consisting of formamide, DMSO, and MTBE.

7. The form of claim 4, wherein:
(a) the form is formed by the recrystallization of a phosphate salt of LSN 487355 in a crystallization solvent comprising nitromethane and THF;
(b) the form is formed by the recrystallization of a phosphate salt of LSN 487355 in a crystallization solvent comprising acetonitrile;
(c) the form is a nitromethane solvate of a phosphate salt of LSN 487355 formed by the recrystallization of the phosphate salt of LSN 487355 in a crystallization solvent comprising nitromethane and THF;
(d) the form is a LSN 487355 mesylate co-crystal formed by the recrystallization of a LSN 487355 salt in a crystallization solvent selected from the group consisting of methanol, methanol and water, and methanol and hydrogen bromide, and the LSN 487355 salt is formed by the reaction of LSN 487355 and methanesulfonic acid;
(e) the form is LSN 509207 Form II obtained by the crystallization of LSN 509207 in a crystallization solvent comprising nitromethane, IPA, and succinic acid;
(f) the form is a LSN 509207 form obtained by the crystallization of LSN 509207 in a crystallization solvent selected from the group consisting of (i) 1,2-dichloroethane and tris base; (ii) acetone; (iii) 1, 2 dichloroethane and either imidazole or sacharrine; and (iv) 1,2-dichloroethane;
(g) the form is a LSN 509207 form obtained by the crystallization of LSN 509207 in a crystallization solvent selected from the group consisting of (i) acetonitrile; (ii) nitromethane, isopropanol, and nicotinamide; and (iii) acetonitrile, isopropanol, and caffeine;
(h) the form is a LSN 509207 form obtained by the crystallization of LSN 509207 in a crystallization solvent selected from the group consisting of (i) n-heptane, 1,2-dichloroethane and acetominophin; and (ii) water, 1,2-dichloroethane and citric acid; or
(i) the form is a LSN 509207 solvate formed by the crystallization of LSN 509207 in a crystallization solvent selected from the group consisting of: methylene chloride, acetone, acetonitrile, nitromethane, THF, 1,4-dioxane, pyridine, and acetic acid.

8. The form of claim 1, comprising an LSN 509207:1-hydroxy-2-naphthoic acid co-crystal formed by the crystallization of LSN 509207 in a crystallization solvent comprising 1,2-dichloroethane.

9. The form of claim 1, comprising an LSN 487355 phosphate salt formed by the crystallization of a LSN 487355 free base in a crystallization solvent comprising either acetonitrile or nitromethane, wherein the LSN 487355 phosphate salt is formed by the reaction of LSN 487355 and phosphoric acid.

10. A pharmaceutical dosage form comprising a pharmaceutically acceptable carrier and a therapeutically effective amount of the form of claim 3.

11. A pharmaceutical dosage form comprising a pharmaceutically acceptable carrier and a therapeutically effective amount of the form of claim 4.

12. A pharmaceutical dosage form comprising a pharmaceutically acceptable carrier and a therapeutically effective amount of the LSN 487355 phosphate salt of claim 9.

13. A method of treatment comprising administering a therapeutically effective amount of the pharmaceutical dosage form of claim 10 to a patient suffering from a neoplasm.

14. A method of treatment comprising administering a therapeutically effective amount of the pharmaceutical dosage form of claim 11 to a patient suffering from a neoplasm.

15. A method of treatment comprising administering a therapeutically effective amount of the pharmaceutical dosage form of claim 12 to a patient suffering from a neoplasm.

16. The method of treatment of claim 13, wherein the neoplasm is a MRP-1-related resistant neoplasm.

17. The method of treatment of claim 14, wherein the neoplasm is a MRP-1-related resistant neoplasm.

18. The method of treatment of claim 15, wherein the neoplasm is a MRP-1-related resistant neoplasm.

19. A method of treatment comprising administering an inhibitory effective amount of the pharmaceutical dosage form of claim 10 to a patient suffering from a MRP-1-related resistant neoplasm.

20. A method of treatment comprising administering an inhibitory effective amount of the pharmaceutical dosage form of claim 111 to a patient suffering from a MRP-1-related resistant neoplasm.

21. A method of treatment comprising administering an inhibitory effective amount of the pharmaceutical dosage form of claim 12 to a patient suffering from a MRP-1-related resistant neoplasm.
